(19) United States
(12) Patent Application Publication
(10) Pub. No.: US 2026/0195986 A1
(43) Pub. Date: Jul. 9, 2026

(54) METHOD AND SYSTEM FOR GENERATING A 3D PARAMETRIC MESH OF AN ANATOMICAL STRUCTURE

(71) Applicant: VITTA Medical Solutions Inc., Montreal (CA)

(72) Inventors: Alessandro SATRIANO, Calgary (CA); Elena DI MARTINO, Calgary (CA); Mitchel BENOVOY, Outremont (CA)

(73) Assignee: VITTA Medical Solutions Inc., Montreal, QC (CA)

(21) Appl. No.: 19/130,592

(22) PCT Filed: Oct. 31, 2023
(86) PCT No.: PCT/IB2023/060971

§ 371 Date: May 16, 2025

Related U.S. Application Data
(60) Provisional application No. 63/383,959, filed on Nov. 16, 2022.

(51) Int. Cl. G06T 17/20; A61B 6/00; G06V 10/26; G06V 10/82

(52) U.S. Cl. CPC G06T 17/20; A61B 6/5217; G06T 2210/41; G06V 10/26; G06V 10/82; G06V 2201/03

(57) ABSTRACT

There is provided a method and a system for generating a 3D parametric mesh of an anatomical structure of a patient for storing multi-domain data therein. A plurality of anatomical segments having been obtained from segmentation of a set of images of a patient are received. A 3D mesh comprising a plurality of concentric 3D mesh layers is received, where each concentric 3D mesh layer includes a same predetermined number of nodes. A set of nodes in the 3D mesh corresponding to a respective anatomical segment is determined to obtain a respective correspondence rule therebetween. The set of nodes is encoded with a set of features from the respective anatomical segment by using the correspondence rule to obtain a 3D parametric mesh, each node of the set of nodes in the 3D parametric mesh being associated with a respective plurality of feature channels comprises the set of features.

### FIELD

The present technology pertains to the field of medical imaging. More precisely, the present technology relates to methods and systems for generating a 3D parametric mesh of an anatomical structure such as an aorta for encoding multidomain features therein.

### BACKGROUND

Postprocessing of vascular imaging helps quantify multiple clinically relevant variables, increasing the diagnostic and prognostic utility of the scan for the reading radiologist (reader) or the referring physician. Specifically, this ensemble of variables generated from the images constitutes a more comprehensive functional assessment of a vessel, thus leading to a more holistic assessment of the patient's health status, and more patient-specific therapeutic approaches and risk assessment.

Quantified variables can come from multiple physics domains such as structural mechanics (e.g., geometry, shape, deformation), fluid dynamics (e.g., luminal flow), or descriptive variables obtained directly from image processing (e.g., image-intensity-related variables). These domains each require processing according to a specific data format, e.g., shell meshes, 3-dimensional solid meshes, or array-like data. These data formats may come with different data-densities, from multiple domains, various patients, and often subsequent scans performed on the same patient. These differences in data format and local data density hinder the ability to systematically report, for each region of the aorta, all available information from the various domains as independent or combined variables.

Further, when training diagnostic or prognostic machine-learning models, the inhomogeneity in data formats forces the data scientist to define multiple models with more parameters. This latter aspect results in more subjects needed to adequately train generalizable diagnostic and predictive models.

### SUMMARY

It is an object of the present technology to ameliorate at least some of the inconveniences present in the prior art. One or more implementations of the present technology may provide and/or broaden the scope of approaches to and/or methods of achieving the aims and objects of the present technology.

One or more implementations of the present technology have been developed based on developers' appreciation that, for prognostic and diagnostic applications, numerous variables or biomarkers need to be obtained from multiple physics domains and descriptive representations, which sometimes require data obtained from multiple imaging modalities, combinations of models, simulations, and other computer processing techniques. Each of these variables may be obtained via different data structures having different data densities and different data formats for regions of the anatomical structures under study. However, these various representations may not be ideal for clinical reporting and machine learning model training.

Developers of the present technology propose a solution that enables homogenizing the data format and the regional data-density across data-domains, between patients, and across patients. The present technology will result in easier multi-domain reporting and easier modelling for machine learning applications.

One or more implementations of the present technology provide an anatomically relevant meshing strategy, yielding homogenized data across multiple modalities and scans. The parametric mesh generated using the present disclosure enables to store data coming from all data types, ranging from shell and solid meshes to array-like data, including pixel-specific data, within stackable layers easily interpretable and utilizable to train more compact machine-learning-based models, relying on a single type of data encoding.

One or more implementations of the present technology provides a three-dimensional (3D) parametric mesh comprising a plurality of concentric 3D mesh layers, where each concentric 3D mesh layer may be interpreted as being a separate 3D mesh representing a different internal or external layer of an anatomical structure of interest, such that multi-domain information on the inside layer (e.g., centerline geometry), the outside layer (e.g., wall geometry, strain) and in-between layers (e.g., blood flow) of the anatomical structure may be stored in the 3D parametric mesh and visually represented.

One or more implementations of the present method and system transform multiple vascular-specific data types in multi-channel, anatomically relevant stackable images that can be used to train diagnostic and prognostic artificial-intelligence-based models, in addition to systematic and intuitive multi-domain reporting to the medical personnel.

One or more implementations of the present technology enable modular modelling for diagnostic and prognostic purposes leveraging each of the domains of available information. Since all models rely on the same datatype, weights can be optionally shared or very minimally re-trained when new information domains are introduced. Modular modelling enables to retrain new architectures, or for new tasks, leveraging on fewer weights (parameters) and requiring the retraining of fewer of these weights. In turn, this facilitates obtaining generalizable models starting from a lower number of vascular scans.

Thus, one or more implementations of the present technology are directed to a method of and a system for generating a parametric mesh of an anatomical structure.

In accordance with a broad aspect of the present technology, there is provided a method for generating a 3D parametric mesh of an anatomical structure for storing multi-domain data therein, the method being executed by at least one processor. The method comprises: receiving a plurality of anatomical segments of at least a portion of an anatomical structure in a body of a given patient having been obtained from segmentation of a set of images having been acquired by a medical imaging apparatus, the set of images comprises at least one image of at least the portion of the anatomical structure in the body of the given patient, receiving a 3D mesh for representing the anatomical structure, the 3D mesh comprises: a plurality of concentric 3D mesh layers, each one of the plurality of concentric 3D mesh layers comprises a same predetermined number of nodes, determining at least one respective set of nodes in the 3D mesh corresponding to at least one respective anatomical segment of the plurality of anatomical segments to obtain a respective correspondence rule therebetween, and encoding, using the correspondence rule, the at least one set of nodes of the 3D mesh with a respective set of features from the at least one respective anatomical segment to obtain a 3D parametric mesh, each node of the at least one set of nodes in the 3D parametric mesh being associated with a respective plurality of feature channels comprises the respective set of features.

In one or more implementations of the method, at least a subset of nodes of the at least one set of nodes are located on different concentric 3D mesh layers.

In one or more implementations of the method, said encoding using the correspondence rule, the at least one set of nodes of the 3D mesh with the respective set of features from the at least one respective anatomical segment to obtain the 3D parametric mesh comprises: determining, using the respective correspondence rule, a respective set of features from biomarkers in the at least one respective anatomical segment, and assigning, to each of the at least one respective set of nodes, the respective set of features from the at least one respective anatomical segment.

In one or more implementations of the method, the method further comprises: receiving a domain representation comprises respective biomarkers related to the anatomical structure in the body of the given patient, determining at least one other respective set of nodes in the 3D parametric mesh corresponding to at least one other region in the domain representation to obtain another respective correspondence rule, at least a subset of the other respective second set of nodes being located on different concentric 3D mesh layers, and encoding, using the other respective correspondence rule, the at least one other respective set of nodes in the 3D parametric mesh with another set of features based on the respective biomarkers, each node of the at least one other respective set of nodes in the 3D parametric mesh being associated with a respective plurality of feature channels comprises the other set of features.

In one or more implementations of the method, each respective node is further associated with at least one time frame for representing the 3D parametric mesh in time.

In one or more implementations of the method, each respective concentric 3D mesh layer is represented as a respective multidimensional array, a location of a given node on the respective 3D mesh layer corresponding to the location of the given node in the respective multidimensional array.

In one or more implementations of the method, the plurality of feature channels for each node of the respective 3D mesh layer is represented as a respective node array, each cell of the respective node array corresponding to a respective feature channel of the plurality of feature channels.

In one or more implementations of the method, the domain representation comprises another mesh different from the 3D parametric mesh, and the another respective correspondence rule comprises determining a mapping between nodes in the another mesh and nodes in the 3D parametric mesh.

In one or more implementations of the method, the another mesh comprises one of: a polygon mesh, the polygon mesh comprises one of a triangle mesh, a quad mesh, a convex polygons mesh, a concave polygons mesh, and a polygon with holes mesh.

In one or more implementations of the method, the domain representation comprises a structural mechanics representation, the respective biomarkers comprise structural mechanics biomarkers, the structural mechanics biomarkers comprises at least one of: pressure values, strain values, and deformation values.

In one or more implementations of the method, the domain representation comprises a fluid dynamics representation, the respective biomarkers comprise at least one of: blood flow values and shear stress values.

In one or more implementations of the method, the domain representation comprises a descriptive variable representation, the respective biomarkers comprise at least one of: geometric data values and image data values.

In one or more implementations of the method, the anatomical structure comprises an aorta of the given patient.

In one or more implementations of the method, the plurality of anatomical segments comprises: a lumen and an aortic wall.

In accordance with a broad aspect of the present technology, there is provided a system for generating a 3D parametric mesh of an anatomical structure for storing multi-domain data therein, the system comprises: at least one processor, and a non-transitory storage medium operatively connected to the at least one processor, the non-transitory storage medium storing computer-readable instructions. The at least one processor, upon executing the computer-readable instructions, being configured for: receiving a plurality of anatomical segments of at least a portion of an anatomical structure in a body of a given patient having been obtained from segmentation of a set of images having been acquired by a medical imaging apparatus, the set of images comprises at least one image of at least the portion of the anatomical structure in the body of the given patient, receiving a 3D mesh for representing the anatomical structure, the 3D mesh comprises: a plurality of concentric 3D mesh layers, each one of the plurality of concentric 3D mesh layers comprises a same predetermined number of nodes, determining at least one respective set of nodes in the 3D mesh corresponding to at least one respective anatomical segment of the plurality of anatomical segments to obtain a respective correspondence rule therebetween, and encoding, using the correspondence rule, the at least one set of nodes of the 3D mesh with a respective set of features from the at least one respective anatomical segment to obtain a 3D parametric mesh, each node of the at least one set of nodes in the 3D parametric mesh being associated with a respective plurality of feature channels comprises the respective set of features.

In one or more implementations of the system, at least a subset of nodes of the at least one set of nodes are located on different concentric 3D mesh layers.

In one or more implementations of the system, said encoding using the correspondence rule, the at least one set of nodes of the 3D mesh with the respective set of features from the at least one respective anatomical segment to obtain the 3D parametric mesh comprises: determining, using the respective correspondence rule, a respective set of features from biomarkers in the at least one respective anatomical segment, and assigning, to each of the at least one respective set of nodes, the respective set of features from the at least one respective anatomical segment.

In one or more implementations of the system, said at least one processor is further configured for: receiving a domain representation comprises respective biomarkers related to the anatomical structure in the body of the given patient, determining at least one other respective set of nodes in the 3D parametric mesh corresponding to at least one other region in the domain representation to obtain another respective correspondence rule, at least a subset of the other respective second set of nodes being located on different concentric 3D mesh layers, and encoding, using the other respective correspondence rule, the at least one other respective set of nodes in the 3D parametric mesh with another set of features based on the respective biomarkers, each node of the at least one other respective set of nodes in the 3D parametric mesh being associated with a respective plurality of feature channels comprises the other set of features.

In one or more implementations of the system, each respective node is further associated with at least one time frame for representing the 3D parametric mesh in time.

In one or more implementations of the system, each respective concentric 3D mesh layer is represented as a respective multidimensional array, a location of a given node on the respective 3D mesh layer corresponding to the location of the given node in the respective multidimensional array.

In one or more implementations of the system, the plurality of feature channels for each node of the respective 3D mesh layer is represented as a respective node array, each cell of the respective node array corresponding to a respective feature channel of the plurality of feature channels.

In one or more implementations of the system, the domain representation comprises another mesh different from the 3D parametric mesh, and the another respective correspondence rule comprises determining a mapping between nodes in the another mesh and nodes in the 3D parametric mesh.

In one or more implementations of the system, the another mesh comprises one of: a polygon mesh, the polygon mesh comprises one of a triangle mesh, a quad mesh, a convex polygons mesh, a concave polygons mesh, and a polygon with holes mesh.

In one or more implementations of the system, the domain representation comprises a structural mechanics representation, the respective biomarkers comprise structural mechanics biomarkers, the structural mechanics biomarkers comprises at least one of pressure values, strain values, and deformation values.

In one or more implementations of the system, the domain representation comprises a fluid dynamics representation, the respective biomarkers comprise at least one of blood flow values and shear stress values.

In one or more implementations of the system, the domain representation comprises a descriptive variable representation, the respective biomarkers comprise at least one of: geometric data values and image data values.

In one or more implementations of the system, the anatomical structure comprises an aorta of the given patient.

In one or more implementations of the system, the plurality of anatomical segments comprises: a lumen and an aortic wall.

### Terms and Definitions

In the context of the present specification, a “server” is a computer program that is running on appropriate hardware and is capable of receiving requests (e.g., from electronic devices) over a network (e.g., a communication network), and carrying out those requests, or causing those requests to be carried out. The hardware may be one physical computer or one physical computer system, but neither is required to be the case with respect to the present technology. In the present context, the use of the expression “a server” is not intended to mean that every task (e.g., received instructions or requests) or any particular task will have been received, carried out, or caused to be carried out, by the same server (i.e., the same software and/or hardware); it is intended to mean that any number of software elements or hardware devices may be involved in receiving/sending, carrying out or causing to be carried out any task or request, or the consequences of any task or request; and all of this software and hardware may be one server or multiple servers, both of which are included within the expressions “at least one server” and “a server”.

In the context of the present specification, “computing device” is any computing apparatus or computer hardware that is capable of running software appropriate to the relevant task at hand. Thus, some (non-limiting) examples of electronic devices include general purpose personal computers (desktops, laptops, netbooks, etc.), mobile computing devices, smartphones, and tablets, and network equipment such as routers, switches, and gateways. It should be noted that a computing device in the present context is not precluded from acting as a server to other computing devices. The use of the expression “a computing device” does not preclude multiple computing devices being used in receiving/sending, carrying out or causing to be carried out any task or request, or the consequences of any task or request, or steps of any method described herein. In the context of the present specification, a “client device” refers to any of a range of end-user client computing devices, associated with a user, such as personal computers, tablets, smartphones, and the like.

In the context of the present specification, unless expressly provided otherwise, a computer system may refer, but is not limited to, an “electronic device”, a “client device”, a “computing device”, an “operation system”, a “system”, a “computer-based system”, a “computer system”, a “network system”, a “network device”, a “controller unit”, a “monitoring device”, a “control device”, a “server”, and/or any combination thereof appropriate to the relevant task at hand.

In the context of the present specification, the expression “computer readable storage medium” (also referred to as “storage medium” and “storage”) is intended to include non-transitory media of any nature and kind whatsoever, including without limitation RAM, ROM, disks (CD-ROMs, DVDs, floppy disks, hard drivers, etc.), USB keys, solid state-drives, tape drives, etc. A plurality of components may be combined to form the computer information storage media, including two or more media components of a same type and/or two or more media components of different types.

In the context of the present specification, a “database” is any structured collection of data, irrespective of its particular structure, the database management software, or the computer hardware on which the data is stored, implemented or otherwise rendered available for use. A database may reside on the same hardware as the process that stores or makes use of the information stored in the database or it may reside on separate hardware, such as a dedicated server or plurality of servers.

In the context of the present specification, the expression “information” includes information of any nature or kind whatsoever capable of being stored in a database. Thus, information includes, but is not limited to audiovisual works (images, movies, sound records, presentations etc.), data (location data, numerical data, etc.), text (opinions, comments, questions, messages, etc.), documents, spreadsheets, lists of words, etc.

In the context of the present specification, unless expressly provided otherwise, an “indication” of an information element may be the information element itself or a pointer, reference, link, or other indirect mechanism enabling the recipient of the indication to locate a network, memory, database, or other computer-readable medium location from which the information element may be retrieved. For example, an indication of a document could include the document itself (i.e., its contents), or it could be a unique document descriptor identifying a file with respect to a particular file system, or some other means of directing the recipient of the indication to a network location, memory address, database table, or other location where the file may be accessed. As one skilled in the art would recognize, the degree of precision required in such an indication depends on the extent of any prior understanding about the interpretation to be given to information being exchanged as between the sender and the recipient of the indication. For example, if it is understood prior to a communication between a sender and a recipient that an indication of an information element will take the form of a database key for an entry in a particular table of a predetermined database containing the information element, then the sending of the database key is all that is required to effectively convey the information element to the recipient, even though the information element itself was not transmitted as between the sender and the recipient of the indication.

In the context of the present specification, the expression “communication network” is intended to include a telecommunications network such as a computer network, the Internet, a telephone network, a Telex network, a TCP/IP data network (e.g., a WAN network, a LAN network, etc.), and the like. The term “communication network” includes a wired network or direct-wired connection, and wireless media such as acoustic, radio frequency (RF), infrared and other wireless media, as well as combinations of any of the above.

In the context of the present specification, the words “first”, “second”, “third”, etc. have been used as adjectives only for the purpose of allowing for distinction between the nouns that they modify from one another, and not for the purpose of describing any particular relationship between those nouns. Thus, for example, it should be understood that, the use of the terms “first server” and “third server” is not intended to imply any particular order, type, chronology, hierarchy or ranking (for example) of/between the servers, nor is their use (by itself) intended to imply that any “second server” must necessarily exist in any given situation. Further, as is discussed herein in other contexts, reference to a “first” element and a “second” element does not preclude the two elements from being the same actual real-world element. Thus, for example, in some instances, a “first” server and a “second” server may be the same software and/or hardware, in other cases they may be different software and/or hardware.

Implementations of the present technology each have at least one of the above-mentioned objects and/or aspects, but do not necessarily have all of them. It should be understood that some aspects of the present technology that have resulted from attempting to attain the above-mentioned object may not satisfy this object and/or may satisfy other objects not specifically recited herein.

Additional and/or alternative features, aspects and advantages of implementations of the present technology will become apparent from the following description, the accompanying drawings and the appended claims.

### BRIEF DESCRIPTION OF THE DRAWINGS

For a better understanding of the present technology, as well as other aspects and further features thereof, reference is made to the following description which is to be used in conjunction with the accompanying drawings, where:

FIG. 1 illustrates a schematic diagram of an electronic device in accordance with one or more non-limiting implementations of the present technology.

FIG. 2 illustrates a schematic diagram of a communication system in accordance with one or more non-limiting implementations of the present technology.

FIG. 3 illustrates a schematic diagram of a parametric mesh generation procedure in accordance with one or more non-limiting implementations of the present technology.

FIG. 4 illustrates a non-limiting example of a perspective view of a visual rendering of a first parametric mesh of an aorta with iliac arteries taken from a front, left side thereof in accordance with one or more non-limiting implementations of the present technology.

FIG. 5A illustrates a perspective view of the visual rendering of the parametric mesh of FIG. 4 with the upper portion removed according to line 11, which shows a plurality of concentric 3D mesh layers.

FIG. 5B illustrates a detailed view of the visual rendering of the parametric mesh of FIG. 5A with a schematic of a selected node and its plurality of feature channels.

FIG. 6 illustrates a top plan view of the visual rendering of the parametric mesh 400 of FIG. 5 with the upper portion removed according to line 11 with a schematic of a selected node and its plurality of feature channels.

FIG. 7 illustrates a perspective view of a visual rendering of a second parametric mesh of the aorta and iliac arteries taken from the front, left side thereof in accordance with one or more non-limiting implementations of the present technology.

FIG. 8 illustrates a schematic diagram of a first user interface showing a visual representation of a third parametric mesh of an aorta with iliac arteries and a corresponding outer layer array with a user interface component for navigating information in the third parametric mesh.

FIG. 9A illustrates a schematic diagram of a second user interface showing a visual representation of a fourth parametric mesh of an aorta with iliac arteries where a concentric mesh layer between the lumen and wall has been selected and a corresponding layer array with user interfaces component for navigating information in the fourth parametric mesh, the second user interface being illustrated in accordance with one or more non-limiting implementations of the present technology.

FIG. 9B illustrates a schematic diagram of the second user interface of FIG. 9A with a core concentric mesh layer selected in the user interface component.

FIG. 10A and FIG. 10B illustrate a flowchart of a method of generating a parametric mesh, the method being executed in accordance with one or more non-limiting implementations of the present technology.

### DETAILED DESCRIPTION

The examples and conditional language recited herein are principally intended to aid the reader in understanding the principles of the present technology and not to limit its scope to such specifically recited examples and conditions. It will be appreciated that those skilled in the art may devise various arrangements which, although not explicitly described or shown herein, nonetheless embody the principles of the present technology and are included within its spirit and scope.

Furthermore, as an aid to understanding, the following description may describe relatively simplified implementations of the present technology. As persons skilled in the art would understand, various implementations of the present technology may be of a greater complexity.

In some cases, what are believed to be helpful examples of modifications to the present technology may also be set forth. This is done merely as an aid to understanding, and, again, not to define the scope or set forth the bounds of the present technology. These modifications are not an exhaustive list, and a person skilled in the art may make other modifications while nonetheless remaining within the scope of the present technology. Further, where no examples of modifications have been set forth, it should not be interpreted that no modifications are possible and/or that what is described is the sole manner of implementing that element of the present technology.

Moreover, all statements herein reciting principles, aspects, and implementations of the present technology, as well as specific examples thereof, are intended to encompass both structural and functional equivalents thereof, whether they are currently known or developed in the future. Thus, for example, it will be appreciated by those skilled in the art that any block diagrams herein represent conceptual views of illustrative circuitry embodying the principles of the present technology. Similarly, it will be appreciated that any flowcharts, flow diagrams, state transition diagrams, pseudo-code, and the like represent various processes which may be substantially represented in computer-readable media and so executed by a computer or processor, whether or not such computer or processor is explicitly shown.

The functions of the various elements shown in the figures, including any functional block labeled as a “processor” or a “graphics processing unit”, may be provided through the use of dedicated hardware as well as hardware capable of executing software in association with appropriate software. When provided by a processor, the functions may be provided by a single dedicated processor, by a single shared processor, or by a plurality of individual processors, some of which may be shared. In some non-limiting implementations of the present technology, the processor may be a general-purpose processor, such as a central processing unit (CPU) or a processor dedicated to a specific purpose, such as a graphics processing unit (GPU). Moreover, explicit use of the term “processor” or “controller” should not be construed to refer exclusively to hardware capable of executing software, and may implicitly include, without limitation, digital signal processor (DSP) hardware, network processor, application specific integrated circuit (ASIC), field programmable gate array (FPGA), read-only memory (ROM) for storing software, random access memory (RAM), and non-volatile storage. Other hardware, conventional and/or custom, may also be included.

Software modules, or simply modules which are implied to be software, may be represented herein as any combination of flowchart elements or other elements indicating performance of process steps and/or textual description. Such modules may be executed by hardware that is expressly or implicitly shown.

With these fundamentals in place, we will now consider some non-limiting implementations of the present technology.

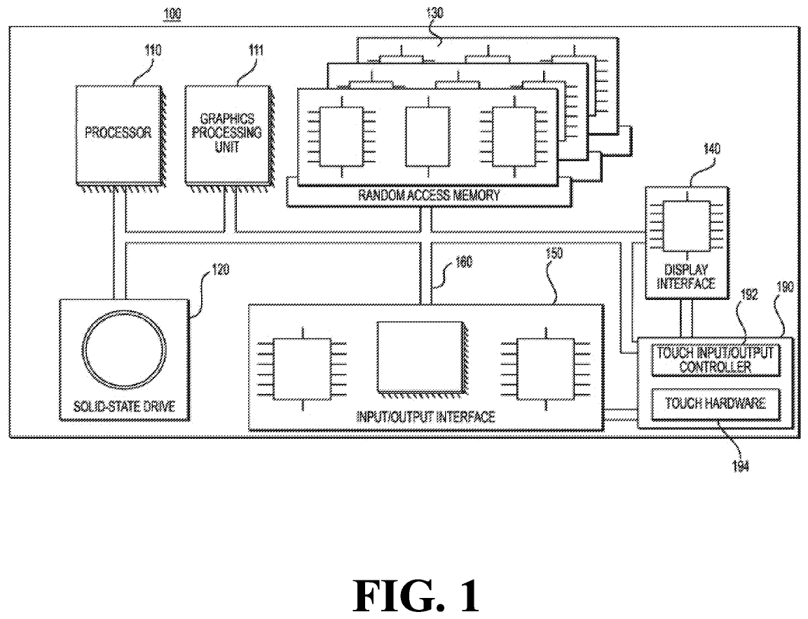

With reference to FIG. 1, there is illustrated a schematic diagram of an computing device 100 suitable for use with some non-limiting implementations of the present technology.

### Computing Device

The computing device 100 comprises various hardware components including one or more single or multi-core processors collectively represented by processor 110, a graphics processing unit (GPU) 111, a solid-state drive 120, a random-access memory 130, a display interface 140, and an input/output interface 150.

Communication between the various components of the computing device 100 may be enabled by one or more internal and/or external buses 160 (e.g. a PCI bus, universal serial bus, IEEE 1394 “Firewire” bus, SCSI bus, Serial-ATA bus, etc.), to which the various hardware components are electronically coupled.

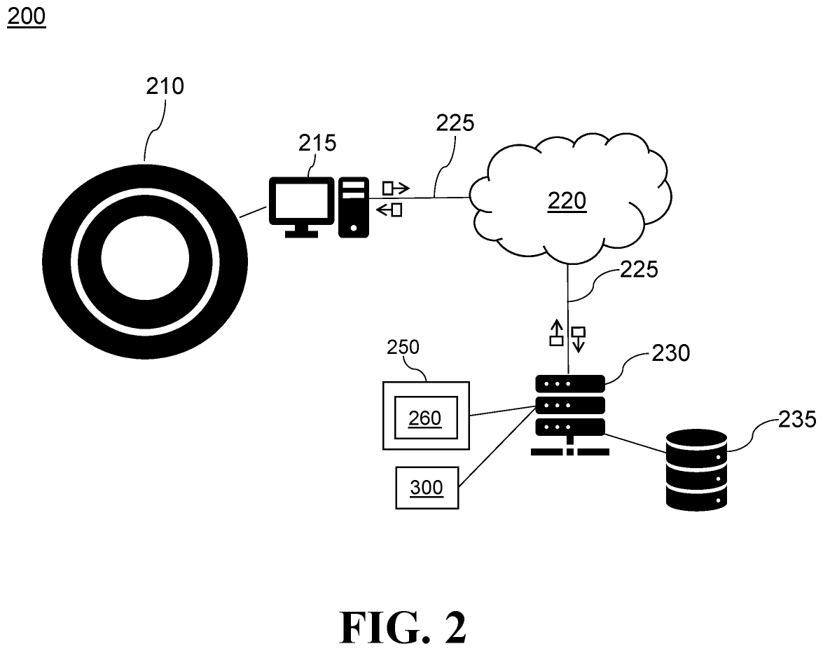

The input/output interface 150 may be coupled to a touchscreen 190 and/or to the one or more internal and/or external buses 160. The touchscreen 190 may be part of the display. In some implementations, the touchscreen 190 is the display. The touchscreen 190 may equally be referred to as a screen 190. In the implementations illustrated in FIG. 2, the touchscreen 190 comprises touch hardware 194 (e.g., pressure-sensitive cells embedded in a layer of a display allowing detection of a physical interaction between a user and the display) and a touch input/output controller 192 allowing communication with the display interface 140 and/or the one or more internal and/or external buses 160. In some implementations, the input/output interface 150 may be connected to a keyboard (not shown), a mouse (not shown) or a trackpad (not shown) allowing the user to interact with the computing device 100 in addition or in replacement of the touchscreen 190.

According to implementations of the present technology, the solid-state drive 120 stores program instructions suitable for being loaded into the random-access memory 130 and executed by the processor 110 and/or the GPU 111 for generating a parametric mesh. For example, the program instructions may be part of a library or an application.

The computing device 100 may be implemented in the form of a server, a desktop computer, a laptop computer, a tablet, a smartphone, a personal digital assistant or any device that may be configured to implement the present technology, as it may be understood by a person skilled in the art.

### System

Referring to FIG. 2, there is shown a schematic diagram of a communication system 200, which will be referred to as the system 200, the system 200 being suitable for implementing non-limiting implementations of the present technology. It is to be expressly understood that the system 200 as illustrated is merely an illustrative implementation of the present technology. Thus, the description thereof that follows is intended to be only a description of illustrative examples of the present technology. This description is not intended to define the scope or set forth the bounds of the present technology. In some cases, what are believed to be helpful examples of modifications to the system 200 may also be set forth below. This is done merely as an aid to understanding, and, again, not to define the scope or set forth the bounds of the present technology. These modifications are not an exhaustive list, and, as a person skilled in the art would understand, other modifications are likely possible. Further, where this has not been done (i.e., where no examples of modifications have been set forth), it should not be interpreted that no modifications are possible and/or that what is described is the sole manner of implementing that element of the present technology. As a person skilled in the art would understand, this is likely not the case. In addition, it is to be understood that the system 200 may provide in certain instances simple implementations of the present technology, and that where such is the case they have been presented in this manner as an aid to understanding. As persons skilled in the art would understand, various implementations of the present technology may be of a greater complexity.

The system 200 comprises inter alia one or more medical imaging apparatuses 210, a server 230 and a database 235 coupled over a communications network 220 via respective communication links 225 (not separately numbered).

In one or more implementations, at least a portion of the system 200 implements the Picture Archiving and Communication System (PACS) technology.

The one or more medical imaging apparatuses 210 are operated by a user (e.g., physician or technician) to acquire medical images of the body of a given patient.

### Medical Imaging Apparatus

The one or more medical imaging apparatuses 210 will now be referred to as the medical imaging apparatus 210.

The medical imaging apparatus 210 is configured to inter alia: (i) acquire, according to acquisition parameters, one or more images of anatomical structures of interest of a given patient; and (ii) transmit the images to the workstation computer 215 and/or the server 230.

The medical imaging apparatus 210 may comprise one of: an X-ray apparatus, a computed tomography (CT) scanner, a magnetic resonance imaging (MRI) scanner, an ultrasound and the like.

In some implementations of the present technology, the medical imaging apparatus 210 may comprise a plurality of medical imaging apparatuses, such as, but not limited to, an X-ray apparatus, a computational tomography (CT) scanner, a magnetic resonance imaging (MRI) scanner, an ultrasound (including 2D or 3D ultrasound), positron emission tomography (PET), single-photon emission computed tomography (SPECT), and the like.

The medical imaging apparatus 210 may be configured with specific acquisition parameters for acquiring images of the patient comprising one or more anatomical structures of interest.

As a non-limiting example, in one or more implementations where the medical imaging apparatus 210 is implemented as a CT scanner, a CT protocol comprising pre-operative retrospectively gated multidetector CT (MDCT—64-row multi-slice CT scanner) with variable dose radiation to capture the R-R interval may be used.

As another non-limiting example, in one or more implementations where the medical imaging procedure comprises a MRI scanner, the MR protocol can comprise steady state T2 weighted fast field echo (TE=2.6 ms, TR=5.2 ms, flip angle 110-degree, fat suppression (SPIR), echo time 50 ms, maximum 25 heart phases, matrix 256×256, acquisition voxel MPS (measurement, phase and slice encoding directions) 1.56/1.56/3.00 mm and reconstruction voxel MPS 0.78

In one or more alternative implementations, the medical imaging apparatus 210 may include or may be connected to a workstation computer 215 for inter alia control of acquisition parameters and image data transmission.

### Workstation Computer

The workstation computer 215 is configured to inter alia: (i) control acquisition parameters of the medical imaging apparatus to perform medical imaging; (ii) receive and process images from the medical imaging apparatus 210; and (iii) transmit the images to the server 230.

The workstation computer 215 is configured to control acquisition parameters of the medical imaging apparatus 210.

The workstation computer 215 may receive images from the medical imaging apparatus 210 in raw format and perform a tomographic reconstruction using known algorithms and software.

The implementation of the workstation computer 215 is known in the art. The workstation computer 215 may be implemented as the computing device 100 or comprise components thereof, such as the processor 110, the graphics processing unit (GPU) 111, the solid-state drive 120, the random-access memory 130, the display interface 140, and the input/output interface 150.

In one implementation, the workstation computer 215 is configured according to the Digital Imaging and Communications in Medicine (DICOM) standard for communication and management of medical imaging information and related data.

The workstation computer 215 is connected to a server 230 over the communications network 220 via a communication link (not numbered).

In one or more alternative implementations, a workstation computer 215 may be provided together with the medical imaging apparatus 210. In one or more other implementations, the workstation computer 215 may be implemented as a mobile device such as a smartphone or a tablet.

In one or more implementations, the medical imaging apparatus 210 is part of a Picture Archiving and Communication System (PACS) for communication and management of medical imaging information and related data together with other electronic devices such as the server 230.

### Server

The server 230 is configured to inter alia: (i) initialize a mesh of one or more anatomical structures according to a set of mesh parameters; (ii) receive a set of images of a given patient comprising at least a portion of the one or more anatomical structures, the set of images having been acquired by the medical imaging apparatus 210; (iii) segment the set of images to obtain a set of segmented images comprising a plurality of anatomical segments; (iv) receive multiple domain representations of the one or more anatomical structures comprising respective biomarkers; (v) determine respective correspondence rules between each domain representation and the initial mesh; and (vi) encode the initial mesh with each of the respective set of features obtained from biomarkers in the domain representation based on the respective correspondence rules to obtain a parametric mesh, each node of the parametric mesh comprising a plurality of feature channels comprising the respective sets of features.

In some implementations, the server 230 has access to the set of machine learning (ML) models 250 to perform some of the aforementioned processes.

How the server 230 is configured to do so will be explained in more detail herein below.

The server 230 can be implemented as a conventional computer server and may comprise some or all of the components of the computing device 100 illustrated in FIG. 2. In an example of one or more implementations of the present technology, the server 230 can be implemented as a Dell™ PowerEdge™ Server running the Microsoft™ Windows Server™ operating system. Needless to say, the server 230 can be implemented in any other suitable hardware and/or software and/or firmware or a combination thereof. In the illustrated non-limiting implementation of present technology, the server 230 is a single server. In alternative non-limiting implementations of the present technology, the functionality of the server 230 may be distributed and may be implemented via multiple servers (not illustrated).

The implementation of the server 230 is well known to the person skilled in the art of the present technology. However, briefly speaking, the server 230 comprises a communication interface (not illustrated) structured and configured to communicate with various entities (such as the workstation computer 215, for example and other devices potentially coupled to the network 220) via the communications network 220. The server 230 further comprises at least one computer processor (e.g., a processor 110 or GPU 111 of the computing device 100) operationally connected with the communication interface and structured and configured to execute various processes to be described herein.

In one or more implementations, the server 230 may be implemented as the computing device 100 or comprise components thereof, such as the processor 110, the graphics processing unit (GPU) 111, the solid-state drive 120, the random-access memory 130, the display interface 140, and the input/output interface 150.

It will be appreciated that the server 230 may provide the output of one or more processing steps to another electronic device for display, confirmation and/or troubleshooting. As a non-limiting example, the server 230 may transmit images, calculated values, results, machine learning parameters, for display on a client device configured similar to the computing device 100 such as a smart phone, tablet, and the like.

The server 230 has access to the set of ML models 250.

### Machine Learning (ML) Models

The set of ML models 250 comprises inter alia a set of segmentation ML models 260.

ML models are referred to as models hereinafter.

Each of the set of models 250 is parametrized by inter alia model parameters and hyperparameters.

The model parameters are configuration variables of the model which are used to perform predictions and estimated or learned from training data, i.e. the coefficients are chosen during learning based on an optimization strategy for outputting a prediction. The hyperparameters are configuration variables of a model which determine the structure of the initial model and how the initial model is trained.

It will be appreciated that the number of model parameters to initialize will depend on inter alia the type of model (e.g., classification or regression), the architecture of the model (e.g., DNN, SVM, etc.), and the model hyperparameters (e.g., a number of layers, type of layers, number of neurons in a NN).

In one or more implementations, the hyperparameters include one or more of: a number of hidden layers and units, an optimization algorithm, a learning rate, momentum, an activation function, a minibatch size, a number of epochs, and dropout.

### Segmentation Model

The set of segmentation models 260 comprise one or more segmentation models.

The segmentation models 260 are configured to perform segmentation of anatomical tissues in images acquired by a medical imaging modality such as the medical imaging apparatus 210.

In one or more implementations, the set of segmentation models 260 is configured to detect all borders (i.e., delimit) and discriminate (i.e., classify) various tissue types in images comprising anatomical structures

In one or more implementations, where the anatomical structures of interest comprise an aortic area, the set of segmentation models 260 is configured to segment the outside wall of the aorta, the inside wall of the aorta, the lumen, and the intraluminal thrombus (ILT). Thus, the segmentation model 260 may classify each pixel in an image as being one of: the outside wall of the aorta, the inside wall of the aorta, the lumen, and the intraluminal thrombus (ILT), and background.

In one or more implementations, the set of segmentation models 260 refers to a plurality of segmentation models 260, each configured to perform a particular segmentation task. As a non-limiting example, the segmentation models 260 may include a first segmentation model configured to perform foreground and background segmentation, a second segmentation model configured to perform semantic segmentation of lumens in aortas, and a third model configured to perform classification of pathological tissues (e.g., classification of calcified versus non-calcified tissues in the aortic wall and intraluminal thrombus (if present)). A non-limiting example of such segmentation models is described in International Patent Application No. PCT/IB2022/051558 entitled “METHOD AND SYSTEM FOR SEGMENTING AND CHARACTERIZING AORTIC TISSUES” filed on Feb. 22, 2022 by the same Applicant, the content of which is incorporated by reference herein.

In one or more implementations, the set of segmentation models 260 may comprises convolutional neural network layers (e.g., U-Net or V-Net based), attention-based mechanisms (i.e., transformer-based models such as a vision transformer (ViT) model) and combinations thereof. It will be appreciated that the set of segmentation models 260 may use encoder-decoder architectures.

In one or more implementations, the set of segmentation models 260 may be based on fully convolutional neural networks (FCNs), generative adversarial networks (GANs), cascaded networks, and the like.

In one or more implementations, the segmentation model 260 has a ResNet-based FCN architecture. Non-limiting examples of ResNet include ResNet50 (50 layers), ResNet101 (101 layers), ResNet152 (152 layers), ResNet50V2 (50 layers with batch normalization), ResNet101V2 (101 layers with batch normalization), and ResNet152V2 (152 layers with batch normalization).

In one or more alternative implementations, the set of segmentation models 260 may be implemented based on one of: U-Net, V-Net, SegNet, AlexNet, GoogleNet, VGG, DeepLab, Mask R-CNN and the like.

### Database

The database 235 is configured to inter alia: (i) store acquisition parameters and data related to the medical imaging apparatus 210; (ii) store images acquired by medical imaging modalities such as the medical imaging apparatus 210; (iii) store data related to the set of ML models 250 including model parameters, hyperparameters, datasets, and outputs; (iv) store data related to meshes; (v) store multiple domain representations including biomarkers; and (vi) parametric meshes including all feature channels thereof.

The database 235 is configured to store images and videos. In one or more implementations, the database may store Digital Imaging and Communications in Medicine (DICOM) files, including for example the DCM and DCM30 (DICOM 3.0) file extensions. Additionally or alternatively, the database 235 may store medical image files in the Tag Image File Format (TIFF), Digital Storage and Retrieval (DSR) TIFF-based format, and the Data Exchange File Format (DEFF) TIFF-based format.

In one or more implementations, the database 235 may store ML file formats, such as .tfrecords, .csv, .npy, and .petastorm as well as the file formats used to store models, such as .pb, .pkl, .pt, or .pth. The database 235 may also store well-known file formats such as, but not limited to image file formats (e.g., .png, .jpeg, exif, .bmp, .tiff), video file formats (e.g., .mp4, .mkv, etc), archive file formats (e.g., zip, .gz, .tar, .bzip2), document file formats (e.g., .docx, .pdf, .txt) or web file formats (e.g., .html).

It will be appreciated that the database 235 may store other types of data such as validation datasets (not illustrated), test datasets (not illustrated) and the like.

### Communication Network

In some implementations of the present technology, the communications network 220 is the Internet. In alternative non-limiting implementations, the communication network 220 can be implemented as any suitable local area network (LAN), wide area network (WAN), a private communication network or the like. It should be expressly understood that implementations for the communication network 220 are for illustration purposes only. How a communication link 225 (not separately numbered) between the workstation computer 215 and/or the server 230 and/or another electronic device (not illustrated) and the communications network 220 is implemented will depend inter alia on how each of the medical imaging apparatus 210, the workstation computer 215, and the server 230 is implemented.

The communication network 220 may be used in order to transmit data packets amongst the workstation computer 215, the server 230 and the database 235. For example, the communication network 220 may be used to transmit requests between the workstation computer 215 and the server 230.

### Parametric Mesh Generation Procedure

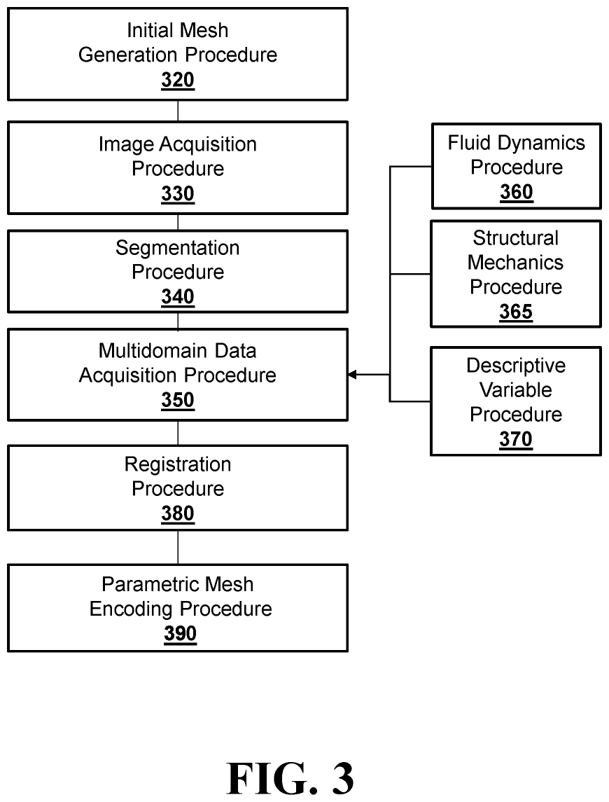

With reference to FIG. 3, there is illustrated a schematic diagram of a parametric mesh generation procedure 300 in accordance with one or more non-limiting implementations of the present technology.

The purpose of the parametric mesh generation procedure 300 is to generate a three-dimensional (3D) parametric mesh, which is a structural representation of one or more anatomical structures of a given patient, where each element or node of the parametric mesh encodes inter alia structural, temporal, functional and other descriptive information, also referred to as biomarkers, at corresponding locations, where the biomarkers may have been obtained using different imaging modalities and/or physics domain representations. How the parametric mesh generation procedure 300 is configured to achieve that purpose is explained in more detail below.

The parametric mesh generation procedure 300 comprises inter alia a mesh initialization procedure 320, an image acquisition procedure 330, a segmentation procedure 340, a multidomain data acquisition procedure 350, a registration procedure 380 and a parametric mesh encoding procedure 500.

In one or more implementations, the parametric mesh generation procedure 300 may be executed by the server 230. In one or more alternative implementations, the parametric mesh generation procedure 300 may be executed by one or more computing devices in a distributed manner. As a non-limiting example, a first computing device such as the server 230 may execute at least a portion of the parametric mesh generation procedure 300 (i.e., one of the procedures) and one or more other computing devices may execute other portions of the parametric mesh generation procedure 300 (i.e., other ones of the procedures).

The parametric mesh generation procedure 300 comprises the mesh initialization procedure 320.

### Mesh Initialization Procedure

The mesh initialization procedure 320 is configured to inter alia: (i) receive a set of mesh parameters; and (ii) generate, based on the set of mesh parameters, an initial 3D mesh. The mesh initialization procedure 320 enables specifying the dimensions, geometry and other attributes of the mesh (e.g. initial conditions or parameters) before the mesh is loaded with patient-specific biomarker data. The mesh initialization procedure 320 serves as a preprocessing step to facilitate the subsequent encoding of the mesh with biomarker data for analysis or visualization.

In the context of the present technology, the mesh initialization procedure 320 is used to initialize a 3D mesh, which is a multidimensional array providing inter alia a spatial representation of one or more anatomical structures in the form of a 3D geometry comprising a discrete number of volumetric elements, also referred to as “elements” or “cells”. It will be appreciated that the mesh may provide a spatial and temporal discrete representation of the one or more anatomical structures for a given patient. The nodes of the initial 3D mesh will then be populated or encoded with single- or multi-modal data for the same patient, i.e., one or more biomarkers, to obtain a 3D parametric mesh, as explained hereinafter. The 3D parametric mesh may be used to store biomarker data from multiple domains in the form of features and visually render the features in time.

The mesh initialization procedure 320 may be executed at any time before the registration procedure 380.

The mesh initialization procedure 320 receives the set of mesh parameters. The set of mesh parameters may be input by an operator of the present technology, for example via an input/output device such as a keyboard, touchscreen, and the like. In one or more implementations, the set of mesh parameters may be received from another client device.

The set of mesh parameters may specify a configuration of the mesh for each anatomical structure represented and may include one or more of its geometry, number of nodes, type of volumetric elements, and number of volumetric elements.

In one or more implementations, the one or more anatomical structures represented by the mesh include an aorta. Additionally, the one or more anatomical structures represented by the mesh may include iliac arteries.

The set of mesh parameters includes a predetermined total number of nodes forming the initial 3D mesh. It will be appreciated that the number of nodes is not limited and may include, as a non-limiting example, 10,000 nodes. In one or more alternative implementations, a number of predetermined feature channels may be associated with each node.

The total number of nodes may be set by an operator of the present technology. In some alternative implementations of the present technology, the number of nodes may depend on a number of the plurality of feature channels, computational resources (e.g., storage capacity of an electronic device used for implementing the present technology) as well as intended use of the 3D parametric mesh.

In one or more implementations, the total number of nodes in the 3D mesh as well as the number of nodes for at least one of each concentric 3D mesh layer, each layer parallel to the axial plane and each layer parallel to the sagittal plane, may also be predetermined.

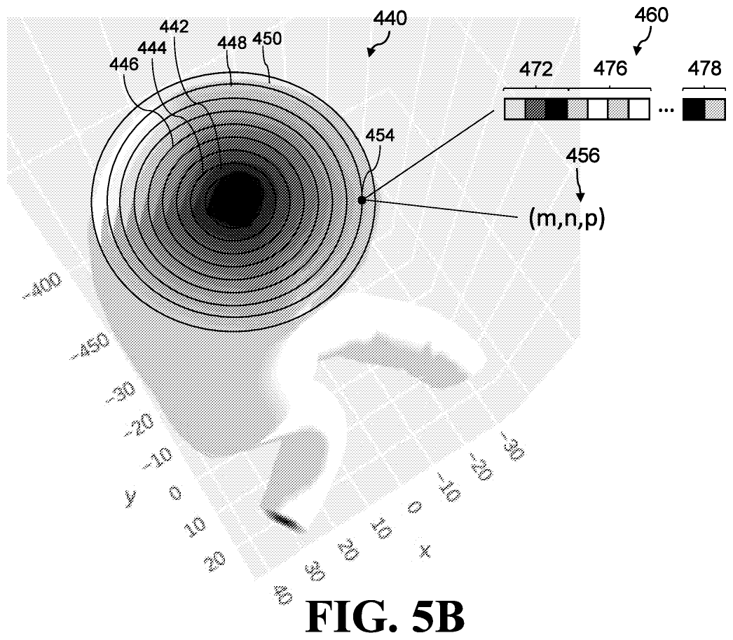

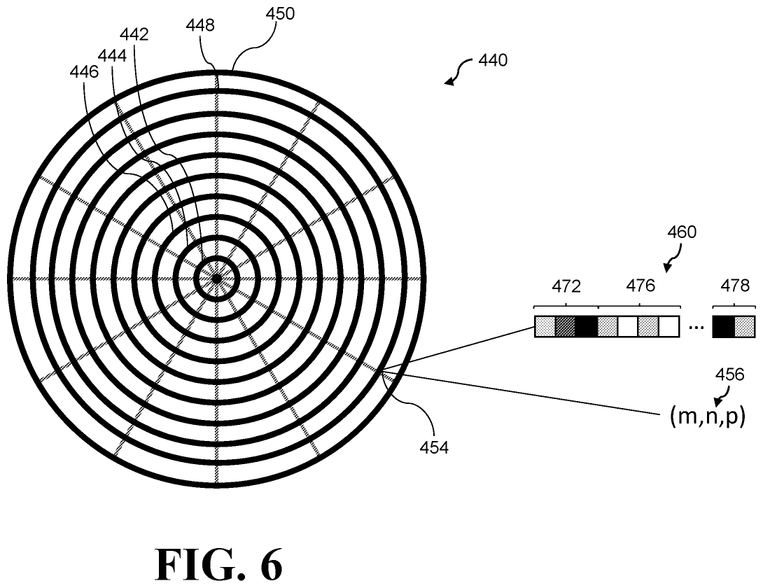

The initial 3D mesh comprises or defines a plurality of concentric 3D mesh layers located relative to a centerline. Each concentric 3D mesh layer may be understood as being a respective 3D mesh with its nodes located at a respective distance from a respective section of a shared centerline (e.g., 3D point or line defining a center of the represented anatomical structure). With brief reference to FIGS. 5A and 5B, there is shown a first parametric mesh 400 with an upper portion removed to show a plurality of concentric 3D mesh layers 440 in FIG. 5B, which is described in more detail below.

Turning back to FIG. 3, the plurality of concentric 3D mesh layers may be used to represent different delimitations of anatomical structures, substructures and/or spaces therebetween where information will be encoded. As a non-limiting example, the initial 3D mesh may comprise 3D mesh layers which may be relative to an outer surface mesh representing an outer surface of the anatomical structure, and/or relative to an innermost mesh at the centerline of the anatomical structure. It will be appreciated that in one or more alternative implementations, the 3D mesh may comprise one or more further layers outside of the anatomical structure.

The centerline of the 3D mesh may correspond to a centerline of the anatomical structure of interest, which will be determined during the registration procedure 380. It should be understood that sections of the centerline may extend vertically and horizontally in 3D, and the centerline generally follows the shape of the anatomical structure being represented.

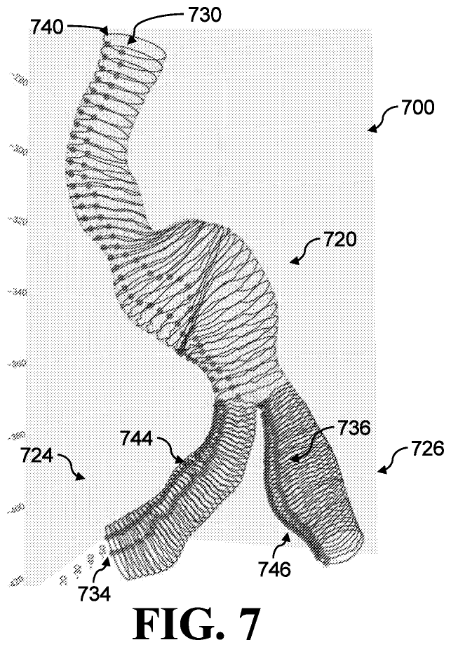

For a given fixed longitudinal coordinate (i.e., fixed vertical or z-axis value) corresponding to an axial (transverse) slice (i.e., parallel to the transverse or axial plane), each concentric 3D mesh layer may also be referred to as a two-dimensional (2D) concentric mesh axial layer or a 2D mesh axial slice. With brief reference to FIG. 6, there is illustrated an axial view of the parametric mesh 400 which shows the plurality of 2D concentric mesh layers 440, which will be described in more detail herein below.

Turning back to FIG. 3, it will be appreciated that when the initial 3D mesh is initialized, its 3D visual representation, has not yet been generated because the initial 3D mesh has not yet been encoded or populated with data from real-life measurements of the anatomical structure(s) of a patient. Thus, the initial 3D mesh is “generic” or the same for each patient, before being encoded with patient-specific data and being optionally visually represented on a graphical user interface. In one or more alternative implementations, the initial 3D mesh may be represented in 3D with a generic or “default” shape of the anatomical structure.

Each concentric 3D mesh layer may be “unwrapped” and represented as a 2D array, where each element in the 2D array corresponds to a respective node in the respective concentric 3D mesh layer. Each node is associated with or represented by a respective node array encoding node features, where the node array may also be referred to as “feature channels”. Each position in the node array corresponds to a respective feature that will be encoded in the respective node of the 3D mesh upon completion of the parametric mesh generation procedure 300.

Each node of the initial 3D mesh is associated with respective node location coordinates. It will be appreciated that the node location coordinates may be expressed in different and equivalent ways depending on the coordinate system used. The node location coordinates are used for referencing the nodes in the mesh or associated array and to store and retrieve information.

In one or more implementations, a given node may be identified based on the concentric 3D mesh layer on which it is located using a mesh layer coordinate R. The mesh layer coordinate R may be used to identify the specific concentric layer array where the node information will be stored, as each concentric 3D mesh layer corresponds to a different array of the same size (i.e., same number of nodes).

In one or more implementations, each node may be identified for each concentric 3D mesh layer, where a coordinate M may refer to the node circumferential position on the concentric 3D mesh layer (i.e., corresponding to its position along the circumference on a 2D mesh axial slice), and a coordinate N may refer to the node longitudinal position (i.e., corresponding to the 2D axial slice the node belongs to). Since each concentric 3D mesh layer may be “unwrapped” and correspond to a 2D array, the node coordinates in the 2D array are the same as the node coordinates in the 3D mesh.

In some implementations, each node of the 3D mesh may be associated with a respective node identifier, which may include one or more numbers for uniquely identifying the node for retrieval and/or storage of information (e.g., in the plurality of feature channels). It will be appreciated that the node coordinates will be the same for each patient.

In one or more implementations, each node is associated with respective node location coordinates and respective node time coordinates. The node time coordinates may include two or more time coordinates that enable representing the nodes at different discrete moments in time. It will be appreciated that the time coordinates (i.e., time frame) may not be required in some implementations of the present technology. Thus, when time coordinates are used, the 3D mesh may be visually represented as it changes in time (e.g., change of geometry of a blood vessel during a cardiac cycle).

In one or more implementations, each concentric 3D mesh layer may comprise M×N×P nodes, where M is a number of circumferential points, N is a number of longitudinal points and P is a number of time frames. It will be appreciated that a plane section (i.e., layer) of the mesh corresponds to all nodes for a fixed longitudinal coordinate N and a fixed time coordinate O.

It will be appreciated that a 3D mesh may be represented as a collection of 2D arrays, where each concentric 3D mesh layer corresponds to a respective 2D array of a same size (i.e., same number of nodes), and where each 2D array element corresponds to a node and includes a respective array corresponding to feature channels for the node. In one or more implementations, time frames may also be encoded in the collection of 2D arrays, where a given collection of 2D arrays (corresponding to a complete 3D mesh). In other implementations, time values may be encoded in each node (i.e., each node may correspond to an array with feature channels for each moment in time).

It should be understood that each node of a 3D mesh of a patient will encode real-life anatomical geometrical information of a patient in the feature channels of the nodes, and the patient-specific 3D mesh of will be rendered visually by using the feature channels encoding the information.

The mesh initialization procedure 320 outputs the initial 3D mesh.

In one or more implementations, the initial 3D mesh is represented as a multidimensional array comprising: a respective concentric layer array representing each concentric 3D mesh layer, where a size of each respective concentric layer array corresponds to a number of nodes in the respective concentric 3D mesh layer, and where each node comprises a respective node array for storing features for the node. Each respective node array may store the node feature at discrete moments in time.

### Image Acquisition Procedure

The image acquisition procedure 330 is configured to inter alia receive a set of images of a body of a patient acquired by the medical imaging apparatus 210.

The set of images of the body of the patient comprises at least one image of the body of a patient, which is a discrete representation of a signal that includes at least a portion of one or more anatomical structures of interest, having been generated using the medical imaging apparatus 210.

It will be appreciated that for 2D domain representations, image cells are referred to as “pixels”, and for 3D domain representations, image cells are referred to as “voxels”.

In one or more implementations, the set of images may be in the form of an image stack.

It will be appreciated that an image stack comprises a set of sequential images, also referred to as slices, that can be scrolled and are expected for cross-sectional studies (e.g., CT/MRI) as well as for time-resolved modalities. As a non-limiting example, an image stack may be provided in the DICOM file format.

In one or more implementations, the image stack may be in the form of a multiphase stack, where each phase of the multiphase stack may correspond to a time instance. As a non-limiting example, each phase in the stack may correspond to a moment in the cardiac cycle of the given patient.

In one or more implementations, the set of images of the body of the patient comprise aorta(s) and/or iliac arteries.

In one or more implementations, the one or more anatomical structures in the set of images may include a thoracic area (e.g., ascending aorta, aortic arch, descending thoracic aorta) and/or abdominal aorta area (e.g., suprarenal abdominal aorta, infrarenal aorta, renal arteries, lumbar arteries) and iliac arteries (e.g., common iliac arteries, external iliac arteries, internal iliac arteries).

It will be appreciated that the image acquisition procedure 330 may receive a plurality of sets of images of the body of the given patient, where each set of images corresponds to a different imaging session. It will be appreciated that a given set of images may be chosen as the “baseline” set and sets of images acquired during other imaging sessions may be transmitted to the multidomain data acquisition procedure 350 and encode subsequently.

In one or more other implementations, the image acquisition procedure 330 may receive a plurality of sets of images, where one or more of the sets of images have been acquired using a different medical imaging apparatus.

The image acquisition procedure 330 outputs the set of images.

### Segmentation Procedure

The segmentation procedure 340 is configured to inter alia: (i) receive the set of images; and (ii) segment the set of images to obtain a set of segmented images comprising a plurality of anatomical segments.

The segmentation procedure 340 uses manual and/or automatic segmentation methods to obtain a plurality of anatomical segments, which may also be referred to as segmented tissues.

In one or more other implementations, the segmentation procedure 340 may use manual segmentation techniques to obtain segmented images.

In one or more implementations, the segmentation procedure 340 uses a set of trained segmentation ML models 260 having been trained to segment anatomical structures in images acquired by an imaging apparatus (e.g., the medical imaging apparatus 210) to obtain a one or more anatomical segments. For example, the segmentation procedure 340 may output for regions in an image, one of a plurality of classes including at least one anatomical segment and a background.

As a non-limiting example, the set of segmentation models 260 may have been trained to segment aortic tissues in images.

In one or more implementations, the segmentation procedure 340 obtains, for each image in the set of images, a segmented aortic area comprising one or more of an aorta and iliac arteries. In one or more implementations, the segmented aortic area comprises a ROI including the lumen, the aortic wall, the ILT (if present), and calcifications (if present).

Additionally, the segmentation procedure 340 may segment different branches of the abdominal aorta including a celiac artery and superior and inferior mesenteric arteries, hepatic artery, splenic artery, renal arteries, and iliac arteries.

In one or more alternative implementations, the segmentation procedure 340 outputs the set of segmented images, where each pixel is categorized with a respective segmented tissue label.

In one or more implementations, the segmentation procedure 340 extracts the segmented tissues from the set of images to obtain at least one image per anatomical segment. It will be appreciated that the segmented tissues may be extracted by performing masking.

In one or more implementations, the segmentation procedure 340 determines a central point for each of the anatomical segments. The central point may be used by the registration procedure 380 to determine a 3D centerline of each anatomical segment.

The segmentation procedure 340 outputs, for each of the set of images, an indication of a plurality of anatomical segments.

### Multidomain Data Acquisition Procedure

The multidomain data acquisition procedure 350 is configured to inter alia: (i) transmit the set of images and/or the set of segmented images comprising the plurality of anatomical segments; and (ii) receive one or more domain representations comprising biomarkers related to the anatomical structure in the body.

In one or more implementations, the multidomain data acquisition procedure 350 transmits the set of images and/or the set of segmented images such that different domain representations may be generated based on the transmitted set of images and/or the plurality of segments. Each domain representation comprises biomarkers related to the anatomical segments.

In the context of the present technology, a domain representation should be understood as being a discretized 2D and/or 3D representation of one or more anatomical structures of interest of a given patient which may or may not include discretized time representations. A domain representation may be computed and/or obtained using one or more imaging modalities.

The domain representation may comprise, or may be associated with, biomarker values which may provide structural, functional and temporal information of the elements comprised in the one or more anatomical structures in the domain representation. Non-limiting example of biomarkers include pixel intensities, pixel positions, structural mechanics values (e.g., pressure, strain, stress, etc.), flow related values (e.g., velocity), and any other descriptive variable.

Each domain representation may have a different data format and/or data density and/or resolution.

In one or more implementations, a given domain representation may be a mesh, such as a polygon mesh. The polygon mesh includes vertices, edges and faces. The faces may include one of: triangles (triangle mesh), quadrilaterals (quads), convex polygons (n-gons), concave polygons, and polygons with holes. The mesh may be a 2D or 3D mesh with or without time discretization.

In one or more other implementations, a given domain representation may be a 2D or 3D image.

In one or more implementations, the multidomain data acquisition procedure 350 transmit the set of images and/or the set of segmented images to a fluid dynamics procedure 360.

### Fluid Dynamics Procedure

The fluid dynamics procedure 360 is configured to perform a fluid dynamics analysis to generate a fluid dynamics representation comprising fluid dynamics biomarkers.

It will be appreciated that that the fluid dynamics procedure 360 may be performed by one or more other electronic devices and/or the server 230.

In one or more implementations, the fluid dynamics procedure 360 uses computational fluid dynamics (CFD) to simulate complex flows by numerical discretization and solution approaches in order to obtain the numerical solution of the discrete time/space points in the flow field. In one or more implementations, the fluid dynamics biomarkers may include heat transfer or fluid flow related variables.

In one or more implementations, the fluid dynamics procedure 360 performs spatial discretization or meshing based on the set of segmented images to divide the geometry into a number of discrete volumetric elements or cells, and then performs temporal discretization. The fluid dynamics procedure 360 sets boundary conditions, i.e., a set of applied physiological parameters (which may vary over time) that define the physical conditions at the inlets, outlets and walls. It will be appreciated that the boundary conditions may be based on patient-specific data, population data, physical models, and/or assumptions. In addition, further properties for the simulation are defined including: blood density and viscosity (i.e., the fluid model), the initial conditions of the system (e.g., whether the fluid is initially static or moving), time discretization information (time step size and numerical approximation schemes), and/or the desired output data (e.g., number of cardiac cycles to be simulated). The fluid dynamics procedure 360 uses a CFD solver to solve the Navier-Stokes and continuity equations, proceeding incrementally towards convergence to obtain a final solution. The fluid dynamics procedure 360 then obtains fluid dynamics biomarkers including a pressure field and velocity field over all elements at each time step. It will be appreciated that additional biomarkers based on the foregoing may be calculated and obtained.

In one or more implementations, the fluid dynamics procedure 360 may use a volumetric mesh ranging between 2 and 3.5 million elements among different possible geometries.

As a non-limiting example, the fluid dynamics procedure 360 may perform sensitivity analysis to obtain a volumetric mesh of approximately 2 million tetrahedral elements and perform CFD simulations in software FLUENT by ANSYS™, by employing Semi-Implicit Method for Pressure Implicit Method for Pressure-Linked Equations (SIMPLE) and a second-order implicit transient formulation, with an assumption of laminar blood flow, and a time varying velocity profile based on flow rate in the descending aorta at the inlet of the fluid domain, with an outflow boundary condition of 50% flow division to the iliac arteries. The rheologic model may assume the blood to be an isotropic, incompressible, Newtonian fluid with assigned constant density (1060 kg/m3) and dynamic viscosity (0.00319 Pa s). The arterial wall may be assumed to be rigid, and no-slip conditions may be applied at the fluid interface. The fluid dynamics procedure 360 may output fluid-dynamics biomarkers for the elements at the boundary of the computational domain. In the preceding non-limiting example, the fluid dynamics procedure 360 may output biomarkers such as blood velocity and wall shear stress at the boundaries of the mesh comprising the 2 tetrahedral million elements.

As a non-limiting example, for the aorta, models such as Windkessel, the distributed model of arterial behavior, reservoir pressure model or finite element analysis may be used to obtain fluid dynamics biomarkers.

The fluid dynamics procedure 360 outputs a fluid dynamics representation comprising the fluid dynamics biomarkers.

The fluid dynamics representation specifies information related to the spatial and temporal discretization, and the fluid dynamics biomarkers specify a number of variables and their values for each of the elements in the spatial and temporal discretization.

The multidomain data acquisition procedure 350 receives the fluid dynamics representation comprising the fluid dynamics biomarkers.

In one or more implementations, the multidomain data acquisition procedure 350 transmit the set of images and/or the set of segmented images to a structural mechanics procedure 365.

### Structural Mechanics Procedure

The structural mechanics procedure 365 is configured to perform a structural mechanics analysis to generate a structural mechanics representation comprising structural mechanics biomarkers. The structural mechanics biomarkers are variables indicative of structural and mechanical properties of the one or more anatomical structures of interest.

It will be appreciated that that the structural mechanics procedure 365 may be performed by one or more other electronic devices and/or the server 230.

The structural mechanics procedure 365 uses the set of segmented images comprising the plurality of anatomical segments to generate a structural mechanics representation which is used to obtain the structural mechanics biomarkers.

The structural mechanics procedure 365 may use a domain representation that is different from other domain representations (e.g., fluid dynamics and descriptive variable representations) to obtain the structural mechanics biomarkers.

In one or more implementations, the structural mechanics procedure 365 performs spatial discretization or meshing based on the set of segmented images to divide the geometry into a number of discrete surface elements or cells, and then performs temporal discretization. The structural mechanics procedure 365 may generate a specific mesh having tetrahedral, triangular, hexahedral, triangular, and/or rectangular elements to compute single- or multi-modal structural mechanics biomarkers. The structural mechanics procedure 365 then performs characterization of the mechanical properties based on the generated mesh. It will be appreciated that different methods may be used, including finite element analysis simulations.

The structural mechanics representation is used to obtain structural mechanics biomarkers, which may include stress-strain relationships and strength of the one or more anatomical structures of interest.

The structural mechanics biomarkers may include thickness, strain, and stress, as well as other biomarkers derived based on the foregoing.

In one or more implementations, the structural mechanics procedure 365 obtains structural mechanics biomarkers including strain. Additionally, the structural mechanics biomarkers may include maximum principal strain, minimum principal strain, circumferential strain, and longitudinal strain, corresponding strain rates, and/or peak strain timing.

As a non-limiting example, the structural mechanics procedure 365 may generate a surface wall mesh of approximately 4000 triangular shell elements and track node velocities on three-dimensional image stacks representing the aorta through the cardiac cycle. The structural mechanics procedure 365 may then measure the nodal displacements based on the node velocities and compute in vivo strain. In this non-limiting example, the structural mechanics procedure 365 uses a domain representation comprising a surface wall mesh of approximately 4000 triangular shell elements, which is different from the initial mesh and the volumetric mesh of approximately 2 million tetrahedral elements used by the fluid dynamics procedure 360.

Thus, this non-limiting example, the structural mechanics procedure 365 uses a domain representation of a surface wall mesh of 4000 triangular elements, and computes strain biomarkers for the elements of the surface wall mesh.

As another non-limiting example, the structural mechanics procedure 365 may use the segmented aortic lumen and the segmented wall received from the segmentation procedure 340 to generate an aortic lumen surface mesh and an aortic wall surface mesh and calculate an ILT thickness by measuring average distance between each mesh point at the outer wall mesh and its neighboring points at the lumen surface within a specified radius.

Non-limiting examples of methods and systems to generate structural mechanics representations and biomarkers, including a regional rupture potential (RRP) of a blood vessel, also referred to as regional aortic weakening (RAW), are described in more detail in International Patent Application Publication WO 2021/059243 A1 entitled “METHOD AND SYSTEM FOR DETERMINING REGIONAL RUPTURE POTENTIAL OF BLOOD VESSEL” filed on Sep. 25, 2020 by the same Applicant, the content of which being incorporated herein by reference.

The structural mechanics representation specifies information related to the spatial and temporal discretization of the anatomical structure(s), and the structural mechanics biomarkers specify a number of variables and their values for each of the elements in the spatial and temporal discretization.

In one or more implementations, the structural mechanics procedure 365 outputs one or more structural mechanics representations each being associated with respective structural biomarkers. Each of the one or more structural mechanics representations may have different mesh geometries and the respective structural biomarkers and thus have different data densities.

The multidomain data acquisition procedure 350 receives the structural mechanics representation comprising the structural mechanics biomarkers.

### Descriptive Variable Representation Procedure

The multidomain data acquisition procedure 350 transmits the set of images and/or the segmented images comprising the plurality of segments to the descriptive variable representation procedure 370 to obtain representations comprising other biomarkers not described above.

In one or more alternative implementations, the multidomain data acquisition procedure 350 may not transmit the images or segments to the descriptive variable representation procedure 370, and obtain one or more descriptive variable representations with biomarkers that are not based on the images or segments. This may be the case for example when other domain representations for the same anatomical structure of the given patient are acquired using other types of imaging modalities

It will be appreciated that that the descriptive variable representation procedure 370 may be performed by one or more other electronic devices and/or the server 230.

In one or more implementations, the descriptive variable representation procedure 370 may receive images of the same patient acquired by one or more other medical imaging apparatus different from the medical imaging apparatus 210.

In one or more implementations, the one or more other medical imaging apparatus may include: micro-CT, Ultrasound, Confocal Microscopy, focused ion beam scanning electron microscopy (FIB SEM), and the like.

It will be appreciated that the descriptive variable representation procedure 370 thus obtains images of at least a portion of the anatomical structures for the same patient.

As a non-limiting example, the descriptive variable representation procedure 370 may receive ultrasound images with Doppler velocities. In this non-limiting example, the ultrasound images may have a different resolution than the set of images acquired by the image acquisition procedure 310 and have a different acquisition angles and views.

The multidomain data acquisition procedure 350 receives one or more descriptive variable representations including respective descriptive biomarkers.

In one or more implementations, the parametric mesh generation procedure 300 includes a registration procedure 380.

### Registration Procedure

The registration procedure 380 is configured to inter alia: (i) receive multiple domain representations from the multidomain data acquisition procedure 350; (ii) align the multiple domain representations into a common frame of reference; and (iii) calculate a respective correspondence rule between the initial mesh and each of the multiple domain representations.

In the context of the present technology, the registration procedure 380 is used to bring the different involved representations and modalities into a common frame of reference (i.e., spatial alignment) so that the information they contain can be optimally integrated in the parametric mesh during the parametric mesh encoding procedure 390.

In one or more implementations, the registration procedure 380 is configured to receive results of a multi-temporal image analysis, when images of the same patient have been acquired at different times and/or under different physical conditions.

In one or more implementations, the registration procedure 380 is configured to perform multimodality image fusion to align images from different modalities acquired by the multidomain data acquisition procedure 350.

In one or more implementations, the registration procedure 380 is configured to perform dynamic image sequence analysis to stack static images that were acquired at different time steps from dynamic image sequences, which are typically used to capture and quantify motion of an anatomy, for example, respiratory/cardiac.

In one or more implementations, the registration procedure 380 is configured to perform data interpolation techniques to obtain biomarker values at locations between cell centers or between cells. Data interpolation techniques may include deterministic and/or statistical interpolation techniques.

Non-limiting examples of data interpolation techniques include nearest neighbor interpolation, linear interpolation, spline interpolation, polynomial interpolation, Lagrange interpolation, Gaussian interpolation, Fourier transforms, and Wavelet transforms.

In one or more implementations, the registration procedure 380 receives additional data used during the multidomain data acquisition procedure 350. As a non-limiting example, when meshes have been generated and modified pre- and postprocessing, the modification information may be transmitted to the registration procedure 380 for easier registration.

In one or more implementations, the registration procedure 380 is configured to determine, for each domain representation of the multiple domain representations, respective center points or a respective centerline of one or more anatomical structures of interest in the domain representation. It will be appreciated that the center points/centerlines may be determined using different methods, including manual methods (e.g., by receiving user inputs), automatic methods or a combination thereof.

The registration procedure 380 is configured to determine a respective correspondence rule between the initial mesh and each of the multiple domain representations. In one or more implementations, the correspondence rule may be determined based on the center points and/or centerline of each domain representation.

The correspondence rule is determined for regions in the domain representation corresponding to regions (i.e., set of nodes) in the initial mesh. As a non-limiting example, the domain representation may be an axial ultrasound image, and the registration procedure 380 may determine the axial view and node coordinates in the mesh that correspond to regions in the axial ultrasound image. The registration procedure 380 then determines a correspondence rule between pixels in the axial ultrasound image and nodes in the mesh.

The respective correspondence rule may include a function mapping element from a given domain representation to corresponding elements (sets of nodes) in the initial 3D mesh, which will enable to map biomarker data associated with the elements of the given domain representation as features on the initial mesh. In other words, each respective correspondence rule is a function describing the mapping between the respective coordinate system of a domain representation to the coordinate system of the initial mesh.

It will be appreciated that since the initial 3D mesh and each of the domain representations may be based on different types of meshes and thus have different biomarker data densities for a given anatomical substructure, there is a need to determine a correspondence between the structural elements, i.e., a location and number of elements in the respective domain representation that corresponds to a given node at a given location in the initial 3D mesh.

The registration procedure 380 calculates a correspondence rule between elements in the initial mesh and the elements in the set of segmented images comprising the plurality of anatomical segments. In one or more implementations, this correspondence rule may be determined first such that the set of segmented images is used as the “baseline” visual representation of the one or more anatomical structures of the patient encoded in the 3D parametric mesh.

The registration procedure 380 calculates a correspondence rule between the elements in the initial mesh and the elements in the structural mechanics representation. As a non-limiting example, the registration procedure 380 may determine regions in the initial mesh of 10,000 nodes corresponding to regions in the surface mesh of 4000 triangular shell elements of the structural mechanics representation and determine the correspondence rule between data associated with the triangular elements and the nodes of the mesh. As a non-limiting example, the regions may include nodes located on different concentric 3D mesh layers.

The registration procedure 380 calculates a respective correspondence rule between the elements in the initial 3D mesh and the elements in the fluid dynamics representation. As a non-limiting example, the registration procedure 380 may determine which regions in the initial mesh of 10,000 nodes correspond to which region in the volumetric mesh of 4,000,000 tetrahedral elements in the fluid dynamics representation and determine the correspondence rule between data associated with the tetrahedral elements and the nodes of the mesh. As a non-limiting example, the regions may include nodes located on different concentric 3D mesh layers.

The registration procedure 380 calculates a respective correspondence rule between the elements in the initial 3D mesh and the elements in the descriptive variable representation. As a non-limiting example, the registration procedure 380 may determine which regions in the initial mesh of 10,000 nodes correspond to which region of pixels in ultrasound images, and determine the correspondence rule between data associated with the pixels and the nodes of the mesh. As a non-limiting example, the regions may include nodes located on different concentric 3D mesh layers.

As a non-limiting example, the registration procedure 380 may determine that a node in the initial mesh corresponds to a plurality of elements pe in the given domain representation. The registration procedure 380, after having determined anchor points, may generate a correspondence rule between the plurality of elements in the given domain representation by specifying that for biomarker data, a weighted average of the biomarkers of pe must be calculated to obtain the feature (i.e., biomarker) value at that node.

Additionally, or alternatively, for elements in a given domain representation that do not correspond directly to nodes on the initial 3D mesh including the plurality of concentric 3D mesh layers, the registration procedure 380 may use a distance to weigh biomarkers values in the correspondence rule.

The registration procedure 380 outputs, for each domain representation a respective correspondence rule.

### Parametric Mesh Encoding Procedure

The parametric mesh encoding procedure 390 is configured to inter alia: (i) receive the initial 3D mesh; (ii) receive multidomain representations from the multidomain data acquisition procedure 350; (iii) receive correspondence rules from the registration procedure 380; (iv) determine, using the correspondence rules, a respective set of features from the respective biomarkers of the multidomain representations for at least one given region; (v) assign the set of features to at least one given region of the initial 3D mesh to obtain a 3D parametric mesh, each node of the at least one region of the parametric mesh being associated with a respective plurality of feature channels comprising at least the set of features.

The purpose of the parametric mesh encoding procedure 390 is to generate, using the initial 3D mesh, a 3D parametric mesh which is a single representation of the anatomical structures of the body of the patient that includes all biomarker data from multiple domain representations. It will be appreciated that biomarker data may be encoded in time on the initial mesh. The 3D parametric mesh may then be provided for display on a user interface and used to render visual representations of the patient specific data be viewed and interacted with, including display of different views in 2D, 3D, and 4D as well as display of multidomain biomarker data encoded within the nodes of the 3D parametric mesh.

### Feature Determination

The parametric mesh encoding procedure 390 is configured to determine, using the respective correspondence rule, for each region in the given domain representation having a corresponding region in the mesh, a respective set of features values from the given domain representation.

The respective set of features corresponds to at least a portion of the biomarkers in the given domain representation. It will be appreciated that the respective set of feature to be extracted may vary depending on the region, concentric mesh layer and domain representation. The features may include all biomarkers from the given domain representation, or only biomarkers of interest from the given domain representation (which may have been predetermined by an operator).

The parametric mesh encoding procedure 390 uses each respective correspondence rule to transform the biomarkers values into node features values that will be assigned to each node in a corresponding region in the initial 3D mesh.

The parametric mesh encoding procedure 390 is configured to encode structural information from the set of segmented images including the plurality of anatomical segments as features in the mesh. The structural information (i.e., positions) of the segments of anatomical structures of the given patient is encoded in the nodes of the initial 3D mesh to obtain the parametric 3D mesh such that it can be used to render a 2D and/or 3D representation of the physical structure of the corresponding anatomical structure as it appears on the set of images for the given patient. The parametric mesh encoding procedure 500 may encode in the parametric 3D mesh structural information such as centerline position, delimitations and positions of substructure of the anatomical structure (e.g., positions of lumen, outer wall, thrombus and calcifications in a segmented aorta). The structural information encoded as features in nodes of the 3D parametric mesh will be used to render the 3D parametric mesh on a user interface. It will be appreciated that the structural information may be encoded in time (when available) such that changes in time of the parametric 3D mesh may also be represented visually (e.g., positions of lumen, outer wall, thrombus and calcifications at different times during the cardiac cycle).

The parametric mesh encoding procedure 390 is configured to encode, using the respective correspondence rule, biomarkers of corresponding elements in the structural mechanics representation into the associated nodes of the mesh. The structural mechanics representation biomarkers are each encoded as a separate feature in the plurality of feature channels of the corresponding node. It will be appreciated that feature positions in the plurality of feature channels may be reserved for the structural mechanics biomarkers, e.g., structural mechanics biomarkers may be encoded in channels 10 to 20 of each node.

The parametric mesh encoding procedure 390 is configured to encode, using the respective correspondence rule, biomarkers of corresponding elements of the fluid mechanics representation into the associated nodes of the mesh. The fluid mechanics representation biomarkers are each encoded as a separate feature in the plurality of feature channels of the corresponding node. It will be appreciated that feature positions in the plurality of feature channels may be reserved for the fluid mechanics biomarkers, e.g., structural mechanics biomarkers may be encoded in channels 21 to 30 of each node.

The parametric mesh encoding procedure 390 is configured to encode, using the respective correspondence rule, biomarkers of corresponding elements in the descriptive variable representation into the associated nodes of the mesh. The descriptive variable representation biomarkers are each encoded as a separate feature in the plurality of feature channels of the corresponding node. It will be appreciated that feature positions in the plurality of feature channels may be reserved for the structural mechanics biomarkers, e.g., structural mechanics biomarkers may be encoded in channels 31 to 50 of each node.

It will be appreciated that all biomarker data of interest for a given region of interest in an anatomical structure may thus be easily stored and retrieved using a single coordinate system of the parametric 3D mesh. Thus, for a given node corresponding to a region in the anatomical structure, e.g., a circumferential point on the ascending aorta, biomarker data from all modalities and physics representation related to that given node may be encoded as features, e.g., position, time, pixel intensities, strain values including maximum principal strain, minimum principal strain, circumferential strain, and longitudinal strain, deformation values, fluid-dynamics data, etc.

The parametric mesh encoding procedure 390 outputs the 3D parametric mesh, which comprises a plurality of concentric 3D mesh layers, each concentric 3D mesh layers having a predetermined number of nodes, with each node comprising a respective plurality of feature channels. The 3D parametric mesh is a patient-specific representation of the anatomical structure of the patient and enables intuitive and systematic reporting of multiple domains of information on an anatomically relevant map extracted from the original vascular scan of the patient.

While the registration procedure 380 and the parametric mesh encoding procedure 390 have been described as separate procedures, it will be appreciated that such a description is for illustrative purposes only, and the registration procedure 380 and the parametric mesh encoding procedure 500 may be combined.

In one or more implementations, the parametric mesh may be stored in a non-transitory storage medium of the server 230 or in the database 235.

In one or more implementations, the parametric mesh may be output and transmitted.

As a non-limiting example, the parametric mesh may be transmitted to the workstation computer 215 for display.

The parametric mesh may be displayed using appropriate 2D or 3D rendering techniques known in the art and interacted with to visualize data from the plurality of feature channels.

The parametric mesh may thus provide, for a given patient, a database that includes all structural, functional, and descriptive data of the anatomical structures of interest. The data, encoded in the form of features at each node location, may thus be quickly retrieved and displayed for analysis.

The parametric mesh generation procedure 300 is repeated for a plurality of patients to obtain respective parametric meshes each encoding all respective biomarkers of each respective patient.

A set of parametric meshes may be used for training different ML models. A given parametric mesh of the set of parametric meshes may be associated with a respective patient.

It will be appreciated that since all ML models rely on the same datatype, weights can be shared or very minimally re-trained when new information domains are introduced. Modular modelling allows to retrain new architectures, or for new tasks, leveraging on less weights (parameters) and requiring the retraining of less of these weights. In turn, this facilitates obtaining generalizable models starting from a lower number of vascular scans.

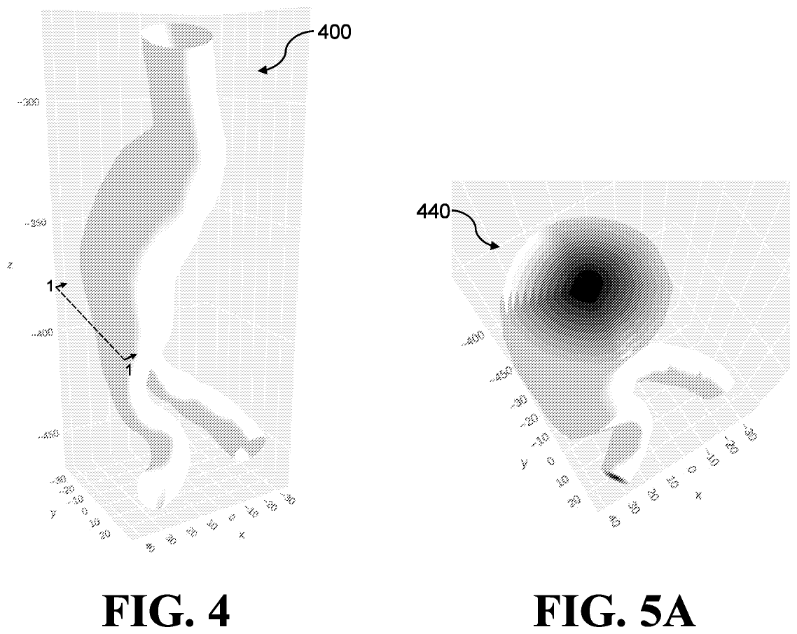

FIG. 4 illustrates a non-limiting example of a perspective view of a rendering of a first parametric mesh 400 of an aorta with iliac arteries taken from a front, left side thereof in accordance with one or more non-limiting implementations of the present technology.

FIG. 5A illustrates a perspective view of the rendering of the first parametric mesh 400 of FIG. 4 with the upper portion removed according to line 11, which shows a plurality of concentric 3D mesh layers 440 in the bottom portion of the first parametric mesh 400.

FIG. 5B illustrates an enlarged and detailed view of the plurality of concentric 3D mesh layers 440 of FIG. 5A with a selected node 454 and its plurality of feature channels 460.

In this illustrated example, the plurality of concentric 3D mesh layers 440 comprises ten layers (not all numbered): a first concentric 3D mesh layer 442, a second concentric 3D mesh layer 444, a third concentric 3D mesh layer 446, . . . , a ninth concentric 3D mesh layer 448, and a tenth concentric 3D mesh layer 450.

The first concentric 3D mesh layer 442 is the innermost layer and represents a core (not numbered) of the first parametric mesh 400.

The tenth concentric 3D mesh layer 450 is the outermost layer and represents the outer wall of the aorta.

As a non-limiting example, the first concentric 3D mesh layer 442 may be used to encode features such as diameter and curvature of the centerline of the aorta, while the tenth layer 450 may be used to encode wall strain, geometry, and presence of calcification, and the concentric layers located in-between, i.e., the second concentric 3D mesh layer 444, the third concentric 3D mesh layer 446, . . . , the ninth concentric 3D mesh layer 448 can be used to encode presence of thrombus, flow patterns, image pixel colours, etc. obtained from the biomarkers in multiple domains (e.g., different imaging modalities, computation fluid dynamics, etc.)

It will be appreciated that while the tenth concentric 3D mesh layer 450 is illustrated as being the outermost layer of the first parametric mesh 400, in one or more other implementations the first parametric mesh 400 may include one or more additional layers located outside of the anatomical structure being represented (e.g., outside of the outer wall of the aorta). Such additional layers may or may not correspond to other anatomical structures and may be used to encode additional information.

FIG. 6 illustrates a top plan view or axial plan view of the rendering of the first parametric mesh 400 of FIG. 5B with the upper portion removed according to line 11.

With reference to FIG. 5B and FIG. 6, each respective node 454 of the ninth concentric 3D mesh layer 448 of the first parametric mesh 400 is associated with respective node coordinates 456 represented by (m, n, p) where m is a circumferential coordinate, n is a longitudinal coordinate and p is a time frame coordinate. Each respective node 454 of the mesh 400 has a plurality of feature channels 460 encoding the features obtained from different biomarkers. The plurality of feature channels 460 may for example include structural features 472, fluid dynamics feature 476, and variable descriptive features 478.

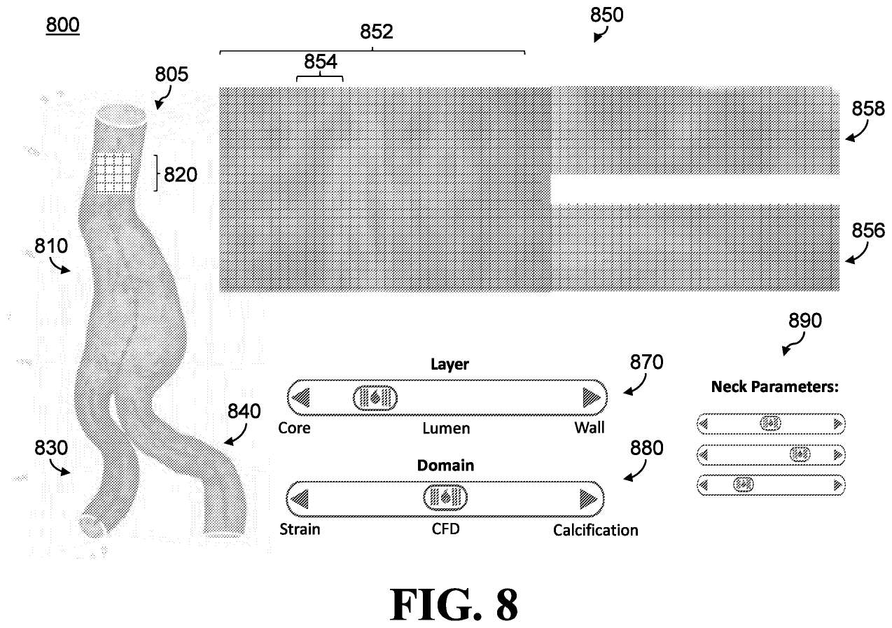

FIG. 7 illustrates a perspective view of a rendering of a second parametric mesh 700 of the aorta and iliac arteries taken from the front, left side thereof in accordance with one or more non-limiting implementations of the present technology.

The second parametric mesh 700 comprises an aorta mesh 720, a left iliac artery mesh 724 and a right iliac artery mesh 726.

In the illustrated second parametric mesh 700, only the outermost or outer surface concentric 3D mesh layer is shown, where each axial layer is represented by an elliptic shape for each discrete axial value (i.e., each longitudinal value). A centerline 730 (corresponding to nodes in the first 3D layer in the aorta mesh 720) extends in the aorta mesh 720 and splits into a left iliac centerline 734 (corresponding to nodes in the first 3D layer of the left iliac artery mesh 724) extending in the left iliac artery mesh 724 and into a right iliac centerline 736 (corresponding to nodes in the first 3D mesh layer in the right iliac artery mesh 726) in the right iliac artery mesh 726.

A second aortic line 740 extends in the aorta mesh 720 and is defined by nodes having the same circumferential coordinate m but a different longitudinal coordinate n (i.e., located on different axial layers). The second aortic line 740 splits into a second left iliac line 744 and a second right iliac line 746. Similarly to the nodes in the second aortic line 740, nodes in each of the second left iliac line 744 and the second right iliac line 746 have the same circumferential coordinate m but a different longitudinal coordinate n in their respective array (i.e., located on different axial layers).

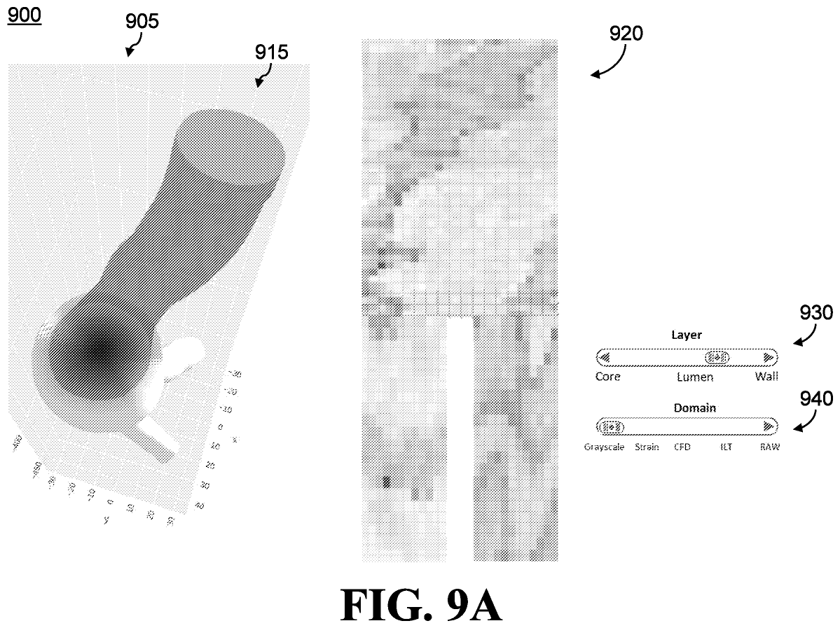

FIG. 8 illustrates a schematic diagram of a first user interface 800 displaying a visual rendering of a third parametric mesh 805, its corresponding third parametric mesh array 850 and user interface elements in the form of a layer slider 870, a domain slider 880 and substructure slider 890.

The third parametric mesh 805 is rendered in 3D on a left side of the user interface 800. The third parametric mesh 805 comprises an aorta mesh 810, a left iliac artery mesh 830 and a right iliac artery mesh 840. It will be appreciated that the visual representation of the third parametric mesh 805 is generated from the structural features encoded in the nodes of the third parametric mesh 805 and visually represents the specific anatomical structure of the given patient for which the multidomain information was extracted.

The third parametric mesh array 850 is displayed on an upper right side of the user interface 800. The third parametric mesh array 850 comprises an aorta mesh array 852, a left iliac artery array 856 and a right iliac artery array 858.

The third parametric mesh array 850 represents the third parametric mesh 805 unwrapped relative to the centerline (or a vertical axis), where columns of the outer layer array 850 correspond to longitudinal node positions (i.e., along the vertical axis) on the third parametric mesh 805 and rows of the third parametric mesh array 850 correspond to circumferential node positions (i.e., along a circumference). Each array element in the third parametric mesh array 850 corresponds to a respective node on the visual rendering of the third parametric mesh 805.

In the non-limiting illustrated example, it can be seen that the neck 820 in the aorta mesh 810 is represented by the neck array 854 within the aorta mesh array 852, while the left iliac artery mesh 830 is represented by the left iliac artery array 856, and the right iliac artery mesh 840 is represented by the right iliac artery array 858.

Each node of the outer layer of the third parametric mesh 805 may be accessed in the third parametric mesh array 850 using the same coordinates (M, N) as above where M is for a circumferential node coordinate and N is for a longitudinal node coordinate. It will be appreciated that each concentric mesh layer of the third parametric mesh 805 may be represented as a respective array of the same size as the third parametric mesh array 850, and each time frame (i.e., corresponding to the parametric mesh 805 at a different moment in time) may be represented as a respective array of the same size.

The layer slider 870 enables to select and display a different concentric 3D mesh layer of the third parametric mesh 805, which includes an innermost core layer, wall layers, and outside layers as well as layers located in between. By selecting a layer using the layer slider 870, structural information encoded in the nodes of that layer may be processed to render a graphical representation of the selected mesh layer.

It will be appreciated that while the graphical rendering displayed on the left changes depending on the selected layer in the layer slider 870, the size and structure of the corresponding third parametric mesh array 850 remains identical, i.e., it has the same number of nodes or elements.

The domain slider 880 enables to select and display a different domain encoded in the third parametric mesh 805, which includes, as a non-limiting example, strain, computation fluid dynamics (CFD) and calcifications. By selecting a domain using the domain slider 880, biomarkers encoded as features in the nodes of may be processed to render a graphical representation of the selected domain. The user interface 800 also comprises the substructure slider 890 in the form of a neck parameter slider which enables displaying different features specific to the neck in the aorta.

While not illustrated in FIG. 8, different types of renderings and projections may be selected by the user using other interface elements.

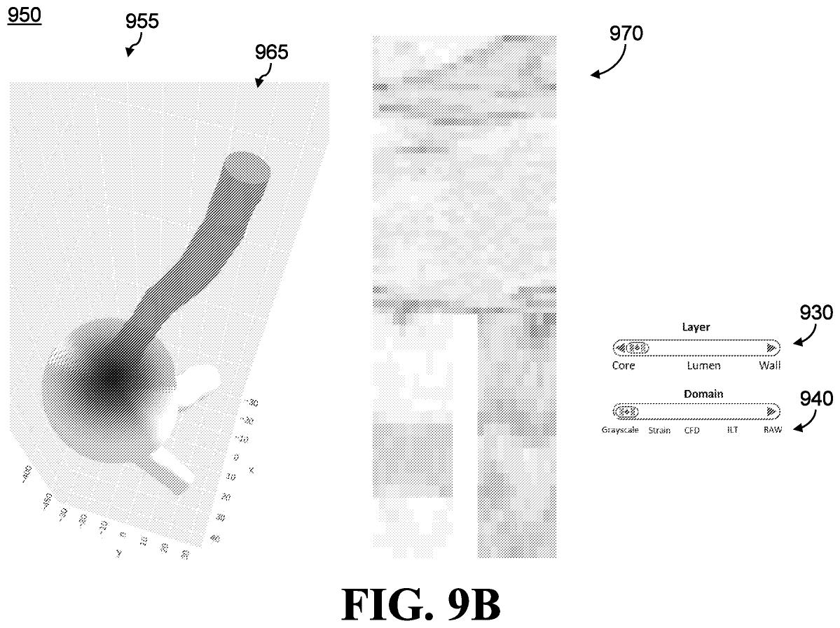

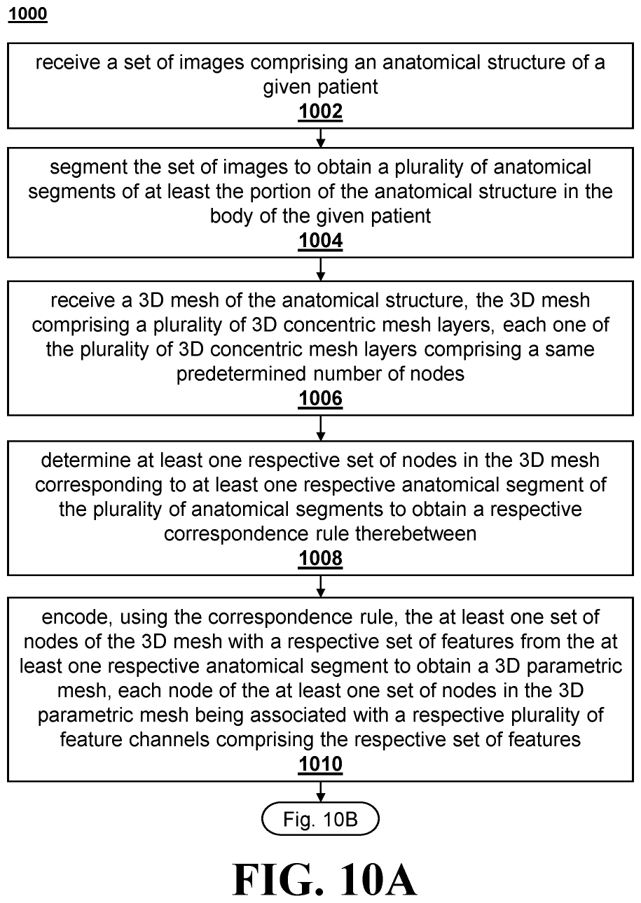

FIG. 9A illustrates a schematic diagram of a second user interface 900 showing a visual representation of a fourth parametric mesh 905 of an aorta with iliac arteries where a concentric mesh layer 915 between the lumen and wall is highlighted after being selected on the layer slider 930, and where the corresponding layer array 920 shows grayscale pixel intensities with the “grayscale” domain being selected on the domain slider 940.

FIG. 9B illustrates a schematic diagram of the second user interface of FIG. 9A where a core concentric mesh layer 965 is highlighted after being selected on the layer slider 930, and where the corresponding layer array 970 shows grayscale pixel intensities for the core concentric mesh layer 965 with the “grayscale” domain being selected on the domain slider 940.

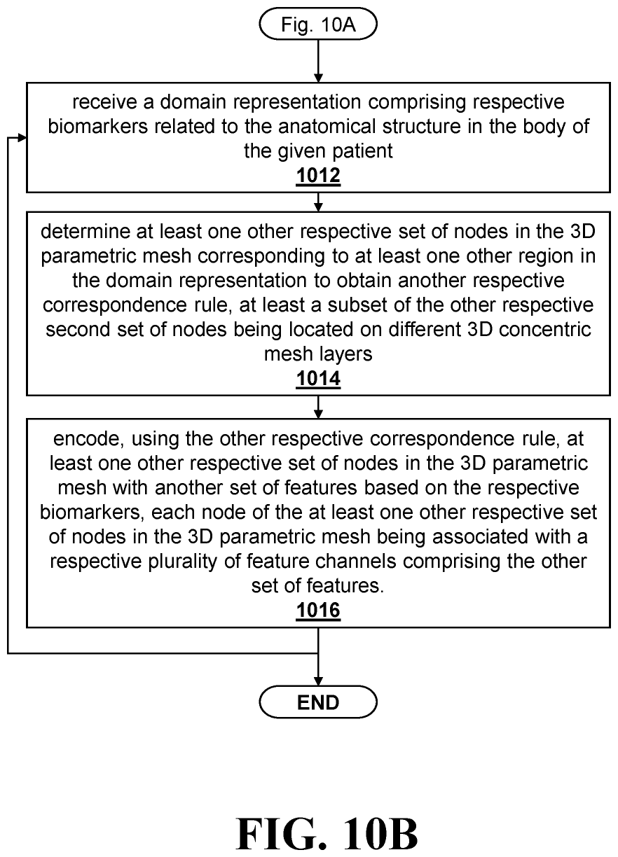

Having described the parametric mesh generation procedure 300 with reference to FIG. 3 and different examples of parametric meshes with reference to FIGS. 4-9B, reference will now be made to FIGS. 10A and 10B, which illustrate a flowchart of a method 1000 of generating a parametric mesh in accordance with one or more non-limiting implementations of the present technology.

It will be appreciated that the procedure(s) in the parametric mesh generation procedure 300 may be integrated into the method 1000.

### Method Description

In one or more implementations, the server 230 comprises at least one processor such as the processor 110 and/or the GPU 111 operatively connected to a non-transitory computer readable storage medium such as the solid-state drive 120 and/or the random-access memory 130 storing computer-readable instructions. The at least one processor, upon executing the computer-readable instructions, is configured to or operable to execute the method 1000.

The method 1000 begins at processing step 1002.

According to processing step 1002, the at least one processor receives a set of images of a given patient having been acquired by a medical imaging apparatus, the set of images comprising at least one image of at least a portion of the anatomical structure in a body of the given patient.

In one or more implementations, the set of images has been acquired by the medical imaging apparatus 210.

In one or more other implementations, the set of images may comprise a plurality of images in the form of an image stack. In one or more alternative implementations, the set of images comprises a plurality of images in the form of a multiphase stack.

In one or more implementations, the anatomical structures include an aorta of the given patient. Additionally, the anatomical structure may include iliac arteries of the given patient.

According to processing step 1004, the at least one processor segments the set of images to obtain a plurality of anatomical segments of at least the portion of the anatomical structure in the body of the given patient.

In one or more implementations, the at least one processor uses manual and/or automatic segmentation methods to obtain the plurality of anatomical segments or segmented tissues.

In one or more implementations, the at least one processor uses a set of trained segmentation models 260 to segment the set of images to obtain the plurality of segments.

As a non-limiting example, the set of trained segmentation models 260 have been trained to segment an aortic area comprising one or more of an aorta and iliac arteries. The segmented aortic area comprises a ROI including the lumen, aortic wall, ILT (if present), and calcifications (if present).

In one or more implementations, processing steps 1002 and 1004 may be replaced by a single processing step where the processor receives the plurality of anatomical segments from another processor.

According to processing step 1006, the at least one processor receives an initial 3D mesh for representing the anatomical structure, the initial 3D mesh comprising: a plurality of concentric 3D mesh layers, each one of the plurality of concentric 3D mesh layers comprising a same predetermined number of nodes.

In one or more implementations, each node has at least one respective time coordinate which enables representing the node and the mesh in time.

The processor initializes a mesh according to a set of mesh parameters specifying at least a geometry and number of nodes of the mesh. Each predetermined region in the mesh may for example be a portion of one or more anatomical structures of interest and may correspond to at least a portion of a segmented anatomical segment obtained by segmentation of the set of images at processing step 1004.

In one or more implementations, processing step 1006 may be executed prior to processing steps 1002 and 1004.

According to processing step 1008, the at least one processor determines at least one region in the mesh corresponding to a given anatomical segment of the plurality of anatomical segments to obtain a correspondence rule between the at least one region in the mesh and the given anatomical segment.

In one or more implementations, the correspondence rule represents a function mapping element from a region in a given anatomical segment of the plurality of anatomical segments to a corresponding region of nodes in the initial mesh.

According to processing step 1010, the at least one processor encodes, using the correspondence rule, the at least one set of nodes of the 3D mesh with a respective set of features from the at least one respective anatomical segment to obtain a 3D parametric mesh, each node of the at least one set of nodes in the 3D parametric mesh being associated with a respective plurality of feature channels comprising the respective set of features.

In one or more implementations, processing step 1010 comprises: determining, using the respective correspondence rule, a respective set of features from biomarkers in the at least one respective anatomical segment, and assigning, to each of the at least one respective set of nodes, the respective set of features from the at least one respective anatomical segment.

In one or more implementations, the at least one processor uses the correspondence rule to extract biomarkers such as positions of the anatomical segments and pixel intensities from the anatomical segment.

It will be appreciated that since the discretized geometry of the initial mesh may differ from the discretized geometry of the plurality of segments in the images, the correspondence rule may specify which region and cell positions in the plurality of segments correspond to a node in the mesh, as well as how to report the values of the cells to the initial 3D mesh. As a non-limiting example, each node may correspond to regions of 4 pixels in the segmented images, and the correspondence rule may thus specify that biomarker values of the 4 pixels must be averaged to obtain a feature. The processor thus determines the features based on the biomarker values for each node based on the correspondence rule, and populates each node with the determines features.

The at least one processor encodes or populates nodes of the mesh with the set of features of the at least one region in the segments. The set of features may for example include positions and pixel intensities. Thus, the 3D parametric mesh provides a discretized 2D or 3D representation of the region in the anatomical segment, which are encoded as node features. In one or more implementations, time information may be encoded as a feature in the nodes of the parametric mesh, which enables visualizing evolution of features in time. The node features may be used to generate a visual representation of the 3D parametric mesh and may also be displayed within one or more arrays corresponding to the 3D parametric mesh.

It will be appreciated that processing steps 1008-1010 may be repeated for other regions and segments such that all required information for the anatomical structures of interest are encoded in the 3D parametric mesh.

According to processing step 1012, the at least one processor receives a domain representation comprising biomarkers related to the anatomical structure in the body of the given patient.

In one or more implementations, the processor receives at least one of a structural mechanics representation comprising structural mechanics biomarkers, a fluid dynamics representation comprising fluid dynamics biomarkers, and a descriptive variable representation comprising variable descriptive biomarkers.

Each domain representation may have a different data format and/or data density and/or resolution.

In one or more implementations, a given domain representation may be a mesh, such as a polygon mesh. The polygon mesh includes vertices, edges, and faces. The faces may include one of triangles (triangle mesh), quadrilaterals (quads), convex polygons (n-gons), concave polygons, and polygons with holes. The mesh may be a 2D or 3D mesh with or without time components.

In one or more other implementations, a given domain representation may be a 2D or 3D image.

In one or more implementations, the processor uses registration techniques to determine the another correspondence rule.

The respective correspondence rule may include a function mapping elements from a given domain representation to corresponding elements in the initial mesh, which will enable to map biomarker data associated with the elements of the given domain representation as features on the initial mesh. In other words, each respective correspondence rule is a function describing the mapping between the respective coordinate system of a domain representation to the coordinate system of the initial mesh.

According to processing step 1014, the processor determines another region in the domain representation corresponding to another given anatomical segment and another region in the parametric mesh to obtain another correspondence rule.

It will be appreciated that the another region may be the same region as in processing step 1008 or a different region.

According to processing step 1016, the processor using the other respective correspondence rule, the at least one other respective set of nodes in the 3D parametric mesh with another set of features based on the respective biomarkers, each node of the at least one other respective set of nodes in the 3D parametric mesh being associated with a respective plurality of feature channels comprising the other set of features.

In one or more implementations, the numbers of features or the features represented in the plurality of feature channels for each node in the 3D parametric mesh may be different.

In one or more implementations, to perform processing step 1016, the processor determines, based on the another correspondence rule, another set of features from the biomarkers related to the given anatomical segment in the domain representation, and assigns, to each node of the at least one other respective set of nodes in the 3D parametric mesh, the respective another set of features.

It will be appreciated that processing steps 1014-1016 may be executed a plurality of times each for a different domain representation comprising respective biomarkers.

The method 1000 then ends.

One or more implementations of the present technology provide an anatomically-relevant meshing strategy, yielding homogenized data across multiple modalities and scans. The parametric mesh generated using the present disclosure enables to store data coming from all data types, ranging from shell and solid meshes to array-like data, including pixel-specific data, within stackable layers easily interpretable and utilizable to train more compact machine-learning-based models, relying on a single type of data encoding.

One or more implementations of the present methods and systems transform multiple vascular-specific data types in multi-channel, anatomically relevant stackable images that can be used to train diagnostic and prognostic artificial-intelligence-based models, in addition to systematic and intuitive multi-domain reporting to the medical personnel.

One or more implementations of the present technology enable modular modelling for diagnostic and prognostic purposes leveraging each of the domains of available information. Since all models rely on the same datatype, weights can be shared or very minimally re-trained when new information domains are introduced. Modular modelling enables to retrain new architectures, or for new tasks, leveraging on fewer weights (parameters) and requiring the retraining of fewer of these weights. In turn, this facilitates obtaining generalizable models starting from a lower number of vascular scans.

In some cases, what are believed to be helpful examples of modifications to the present technology may also be set forth. This is done merely as an aid to understanding, and, again, not to define the scope or set forth the bounds of the present technology. These modifications are not an exhaustive list, and a person skilled in the art may make other modifications while nonetheless remaining within the scope of the present technology. Further, where no examples of modifications have been set forth, it should not be interpreted that no modifications are possible and/or that what is described is the sole manner of implementing that element of the present technology.

Modifications and improvements to the above-described implementations of the present technology may become apparent to those skilled in the art. The foregoing description is intended to be exemplary rather than limiting.

1. A method for generating a 3D parametric mesh of an anatomical structure for storing multi-domain data therein, the method being executed by at least one processor, the method comprising:
receiving a plurality of anatomical segments of at least a portion of an anatomical structure in a body of a given patient having been obtained from segmentation of a set of images having been acquired by a medical imaging apparatus, the set of images comprising at least one image of at least the portion of the anatomical structure in the body of the given patient;
receiving a 3D mesh for representing the anatomical structure, the 3D mesh comprising:
a plurality of concentric 3D mesh layers, each one of the plurality of concentric 3D mesh layers comprising a same predetermined number of nodes;
determining at least one respective set of nodes in the 3D mesh corresponding to at least one respective anatomical segment of the plurality of anatomical segments to obtain a respective correspondence rule therebetween; and
encoding, using the correspondence rule, the at least one respective set of nodes of the 3D mesh with a respective set of features from the at least one respective anatomical segment to obtain a 3D parametric mesh, each node of the at least one respective set of nodes in the 3D parametric mesh being associated with a respective plurality of feature channels comprising the respective set of features.

2. The method of claim 1, wherein at least a subset of nodes of the at least one set of nodes are located on different concentric 3D mesh layers.

3. The method of claim 1 or 2, wherein said encoding using the correspondence rule, the at least one set of nodes of the 3D mesh with the respective set of features from the at least one respective anatomical segment to obtain the 3D parametric mesh comprises:
determining, using the respective correspondence rule, a respective set of features from biomarkers in the at least one respective anatomical segment; and
assigning, to each of the at least one respective set of nodes, the respective set of features from the at least one respective anatomical segment.

4. The method of claim 1, further comprising:
receiving a domain representation comprising respective biomarkers related to the anatomical structure in the body of the given patient;
determining at least one other respective set of nodes in the 3D parametric mesh corresponding to at least one other region in the domain representation to obtain another respective correspondence rule, at least a subset of the other respective second set of nodes being located on different concentric 3D mesh layers; and
encoding, using the other respective correspondence rule, the at least one other respective set of nodes in the 3D parametric mesh with another set of features based on the respective biomarkers, each node of the at least one other respective set of nodes in the 3D parametric mesh being associated with a respective plurality of feature channels comprising the other set of features.

5. The method of claim 1, wherein each respective node is further associated with at least one time frame for representing the 3D parametric mesh in time.

6. The method of claim 1, wherein each respective concentric 3D mesh layer is represented as a respective multidimensional array, a location of a given node on the respective 3D mesh layer corresponding to the location of the given node in the respective multidimensional array.

7. (canceled)

8. The method of claim 2, wherein the domain representation comprises another mesh different from the 3D parametric mesh, and wherein the another respective correspondence rule comprises determining a mapping between nodes in the another mesh and nodes in the 3D parametric mesh.

9. (canceled)

10. The method of claim 2, wherein
the domain representation comprises a structural mechanics representation; and wherein
the respective biomarkers comprise structural mechanics biomarkers, the structural mechanics biomarkers comprising at least one of: pressure values, strain values, and deformation values.

11. The method of claim 2, wherein the domain representation comprises a fluid dynamics representation; and wherein the respective biomarkers comprise at least one of: blood flow values and shear stress values.

12. The method of claim 2, wherein
the domain representation comprises a descriptive variable representation; and wherein
the respective biomarkers comprise at least one of: geometric data values and image data values.

13. (canceled)

14. (canceled)

15. A system for generating a 3D parametric mesh of an anatomical structure for storing multi-domain data therein, the system comprising:
at least one processor; and
a non-transitory storage medium operatively connected to the at least one processor, the non-transitory storage medium storing computer-readable instructions; and
the at least one processor, upon executing the computer-readable instructions, being configured for:
receiving a plurality of anatomical segments of at least a portion of an anatomical structure in a body of a given patient having been obtained from segmentation of a set of images having been acquired by a medical imaging apparatus, the set of images comprising at least one image of at least the portion of the anatomical structure in the body of the given patient;
receiving a 3D mesh for representing the anatomical structure, the 3D mesh comprising:
a plurality of concentric 3D mesh layers, each one of the plurality of concentric 3D mesh layers comprising a same predetermined number of nodes;
determining at least one respective set of nodes in the 3D mesh corresponding to at least one respective anatomical segment of the plurality of anatomical segments to obtain a respective correspondence rule therebetween; and
encoding, using the correspondence rule, the at least one respective set of nodes of the 3D mesh with a respective set of features from the at least one respective anatomical segment to obtain a 3D parametric mesh, each node of the at least one respective set of nodes in the 3D parametric mesh being associated with a respective plurality of feature channels comprising the respective set of features.

16. The system of claim 15, wherein at least a subset of nodes of the at least one set of nodes are located on different concentric 3D mesh layers.

17. The system of claim 15, wherein said encoding using the correspondence rule, the at least one set of nodes of the 3D mesh with the respective set of features from the at least one respective anatomical segment to obtain the 3D parametric mesh comprises:
determining, using the respective correspondence rule, a respective set of features from biomarkers in the at least one respective anatomical segment; and
assigning, to each of the at least one respective set of nodes, the respective set of features from the at least one respective anatomical segment.

18. The system of claim 15, wherein said at least one processor is further configured for:
receiving a domain representation comprising respective biomarkers related to the anatomical structure in the body of the given patient;
determining at least one other respective set of nodes in the 3D parametric mesh corresponding to at least one other region in the domain representation to obtain another respective correspondence rule, at least a subset of the other respective second set of nodes being located on different concentric 3D mesh layers; and
encoding, using the other respective correspondence rule, the at least one other respective set of nodes in the 3D parametric mesh with another set of features based on the respective biomarkers, each node of the at least one other respective set of nodes in the 3D parametric mesh being associated with a respective plurality of feature channels comprising the other set of features.

19. The system of claim 15, wherein each respective node is further associated with at least one time frame for representing the 3D parametric mesh in time.

20. The system of claim 15, wherein each respective concentric 3D mesh layer is represented as a respective multidimensional array, a location of a given node on the respective 3D mesh layer corresponding to the location of the given node in the respective multidimensional array.

21. (canceled)

22. The system of claim 16, wherein the domain representation comprises another mesh different from the 3D parametric mesh, and wherein the another respective correspondence rule comprises determining a mapping between nodes in the another mesh and nodes in the 3D parametric mesh.

23. (canceled)

24. The system of claim 16, wherein
the domain representation comprises a structural mechanics representation; and wherein
the respective biomarkers comprise structural mechanics biomarkers, the structural mechanics biomarkers comprising at least one of: pressure values, strain values, and deformation values.

25. The system of claim 16, wherein
the domain representation comprises a fluid dynamics representation; and wherein
the respective biomarkers comprise at least one of: blood flow values and shear stress values.

26. The system of claim 16, wherein
the domain representation comprises a descriptive variable representation; and wherein
the respective biomarkers comprise at least one of: geometric data values and image data values.

27. (canceled)

28. (canceled)
